(19) United States
(12) Patent Application Publication
(10) Pub. No.: US 2023/0380915 A1
(43) Pub. Date: Nov. 30, 2023

(54) ROBOTIC DRIVE SYSTEM FOR FACILITATING TREATMENTS OF THE NEUROVASCULATURE AND METHODS OF USE

(71) Applicant: Route 92 Medical, Inc., San Mateo, CA (US)

(72) Inventors: Ronald R. Hundertmark, San Mateo, CA (US); John Miller, San Mateo, CA (US); Tony M. Chou, San Mateo, CA (US)

(21) Appl. No.: 18/323,756

(22) Filed: May 25, 2023

Related U.S. Application Data
(60) Provisional application No. 63/346,733, filed on May 27, 2022.

(51) Int. Cl. A61B 34/30; A61M 25/09; A61B 34/20

(52) U.S. Cl. CPC A61B 34/30; A61M 25/09041; A61B 34/20; A61B 2034/301; A61M 2025/091; A61B 2034/2046

(57) ABSTRACT

A robotic procedure system for treating neurovasculature of a patient including a guide sheath, catheter system having a support catheter and a navigation catheter, and robotic drive system configured to drive the catheter system within a patient's vessel. The robotic drive system includes a cassette with at least a first set of rollers and at least a second set of rollers and a controller operatively coupled to the cassette. The first set of rollers is configured to engage a proximal control element of the support catheter and the second set of rollers configured to engage a proximal extension of the navigation catheter. The controller is configured to control the first set and second set of rollers so as to determine a magnitude of linear translation of the support catheter and a magnitude of linear translation of the navigation catheter. Related devices, systems, and methods are provided.

### CROSS-REFERENCE TO RELATED APPLICATIONS

This application claims the benefit of priority under 35 U.S.C. § 119(e) to U.S. Provisional Patent Application Ser. No. 63/346,733, filed May 27, 2022. The disclosure is hereby incorporated by reference in its entirety.

### FIELD

The present disclosure relates generally to the field of catheter procedure systems and, in particular, robotic systems and methods for automated movement of elongated medical devices configured to be advanced into the neurovasculature for the treatment of intracranial pathologies, such as occlusions in acute ischemic stroke, ICAD, aneurysms, and other intracranial pathologies.

### BACKGROUND

Interventions are performed via catheter-based systems to treat various vascular diseases, including neurovascular intervention. Vessel stenoses or intracranial atherosclerotic disease (ICAD) can be treated by endovascular implantation of scaffolding devices, such as stents, often in combination with balloon angioplasty, to increase the inner diameter or cross-sectional area of the vessel lumen. Other vascular defects include aneurysms in which a bulge or bubble protrudes out in a radial direction from the vessel that, if left untreated, may continue expanding until it bursts thereby causing hemorrhaging from the vessel. Still further, occlusions in the intracranial vessels leading to Acute Ischemic Stroke (AIS) or the sudden blockage of adequate blood flow to a section of the brain, can be treated endovascularly, such as by the delivery large-bore catheters for delivery of aspiration embolectomy and/or retrievable stent devices to aid in removal of the clot.

Treatment of the vessels of the brain is particularly challenging due, in part, to the tortuosity of the vasculature and the small size of the vessels. Further, the risk of stroke and thromboembolic complications is high due to the release of thrombotic material during delivery and treatment. The internal carotid artery (ICA) arises from the bifurcation of the common carotid artery at the level of the intervertebral disc between C3 and C4 vertebrae. The course of the ICA is divided into four parts—cervical, petrous, cavernous and cerebral parts. In the anterior circulation, the consistent tortuous terminal carotid is locked into its position by bony elements. The cervical carotid enters the petrous bone and is locked into a set of turns as it is encased in bone. The cavernous carotid is an artery that passes through a venous bed, the cavernous sinus, and while flexible, is locked as it exits the cavernous sinus by another bony element, which surrounds and fixes the entry into the cranial cavity. Because of these bony points of fixation, the petrous carotid and above are relatively consistent in their tortuosity. The carotid siphon S (see FIG. 1B) is an S-shaped part of the terminal ICA. The carotid siphon S begins at the posterior bend of the cavernous ICA and ends at the ICA bifurcation into the anterior cerebral artery and middle cerebral artery. The ophthalmic artery arises from the cerebral ICA, which represents a common point of catheter hang up in accessing the anterior circulation. These points of catheter hang up can significantly increase the amount of time needed to access the vessels of the brain. Where the procedure involves restoring blood perfusion to the brain, added time and difficulty of catheter system navigation is a clear disadvantage with severe consequences.

Robotically controlled systems allow clinicians to deliver various surgical tools to locations within a patient's body. There is a need for robotically controlled systems for the delivery of neurointerventional catheter systems to aid in the navigation of catheter systems to distal sites in the brain, particularly systems for catheters that are designed to navigate the challenging anatomy of the brain while also improving delivery of aspiration forces to distal sites.

### SUMMARY

In an aspect, provided is a robotic procedure system for treating neurovasculature of a patient. The system includes a guide sheath having a sheath body with at least one lumen extending between a proximal end region and a distal end region defining a distal opening from the at least one lumen; and a hub coupled to the proximal end region of the sheath body. The system includes a catheter system including a support catheter having a distal luminal portion having an outer diameter sized to be positioned within the at least one lumen of the guide sheath, the distal luminal portion coupled at a proximal end region to a proximal control element adjacent a proximal opening from a single lumen of the distal luminal portion; and a navigation catheter having a guidewire lumen, a distal tip region that tapers from an outer diameter sized to fill the single lumen of the support catheter to a distal-most end defining an opening from the guidewire lumen, and a proximal extension The system includes a robotic drive system configured to drive the catheter system within a patient's vessel. The robotic drive system includes a cassette having at least a first set of rollers and at least a second set of rollers. The first set of rollers is configured to engage the proximal control element of the support catheter and the second set of rollers is configured to engage the proximal extension of the navigation catheter. The second set of rollers is positioned proximal of the first set of rollers. The system includes a controller operatively coupled to the cassette. The controller is configured to control the first set and second set of rollers so as to determine a magnitude of linear translation of the support catheter and a magnitude of linear translation of the navigation catheter.

The system can further include a guidewire and a third set of rollers located proximal to the second set of rollers that is configured to engage the guidewire. The spacing of the third set of rollers can be designed to allow a full range of motion of the navigation catheter through the second set of rollers. The proximal control element of the support catheter can be a ribbon, a hypotube, or a solid round wire. The proximal extension of the navigation catheter can be a polymer-coated rigid component. At least one of the first and second set of rollers can be configured to accommodate different outer diameters. Rollers of the first set of rollers can be spaced closer together than rollers of the second set of rollers. The first set of rollers can be proximal to and off-set from an axis of the guide sheath working lumen.

The system can further include an aspiration system operatively coupled to the controller. The aspiration system can be configured to apply static or cyclic aspiration. The cyclic aspiration can be applied using a spring-operated pressure relief valve or a solenoid valve. The aspiration system can be actuated manually or by software running on the controller. The guide sheath can be coupled to the robotic drive system by securing the hub to the cassette via at least one connector and/or cavity within the cassette. The at least one connector can be configured to rotate the guide sheath around a longitudinal axis of the sheath body. At least one of the first set and the second set of rollers can be configured change the magnitude of linear translation, an angle, or both. The first set of rollers and the second set of rollers can be configured to be driven in unison. The first set of rollers and the second set of rollers can be driven in unison due to a mechanical linkage.

The system can include one or more markers on the support catheter and/or one or more markers on the navigation catheter. The controller can be programmed to detect the one or more markers on the support catheter and the one or more markers on the navigation catheter to assess extension of the support catheter relative to the navigation catheter. The controller can be programmed to detect the one or more markers on the support catheter and the one or more markers on the navigation catheter to assess total distance of advancement. The system can include one or more flow sensors and one or more pressure transducers. The system can further include an oscillation input configured to cause the support catheter to follow a pattern of one or more retractions and one or more advancements. The oscillation input can be programmable by a user. The oscillation input can initiate a pattern of a short retraction of the support catheter withdrawing a distal opening of the distal luminal portion from a first position relative to an occlusion to a second position relative to the occlusion and an advancement of the support catheter advancing the distal opening from the second position towards the first position. The pattern can begin after a period of static aspiration through the support catheter.

In an interrelated aspect, provided is a method for performing robotic surgery on a patient in the neurovasculature. The method includes coupling a catheter system to a robotic drive system, the robotic drive system operatively coupled to a controller operable by inputs from an operator, the robotic drive system having a plurality of rollers movable in response to operator inputs. The catheter system includes a support catheter having a distal luminal portion with a distal opening and a proximal opening, a single lumen extending between the proximal opening and the distal opening; and a proximal control element without a lumen coupled to the distal luminal portion near the proximal opening; and a navigation catheter including a guidewire lumen; an outer diameter sized to fill the single lumen of the support catheter; a distal tip region; and a proximal extension. The navigation catheter is axially positionable through the single lumen of the support catheter so that the distal tip region of the navigation catheter is extendable from the distal opening of the distal luminal portion of the support catheter. The support catheter having the navigation catheter positioned within the single lumen is navigable to a vessel distal to a petrous portion of an internal carotid artery. The method includes coupling the proximal control element of the support catheter to a first set of rollers of the plurality of rollers; coupling the proximal extension of the navigation catheter to a second set of rollers of the plurality of rollers, the second set of rollers located proximal of the first set of rollers; and moving the first and second sets of navigation rollers in at least one degree of freedom in response to operator inputs.

In an interrelated aspect, provided is a robotically controlled procedure system for removing a clot from a patient. The system includes a guide sheath having at least one working lumen; a catheter system including a catheter having a distal luminal portion with an outer diameter sized to be positioned within the working lumen, the distal luminal portion coupled at a proximal end region to a proximal control element adjacent a proximal opening from a single lumen of the distal luminal portion. The guide sheath is operatively coupled to an aspiration system for performing an aspiration function on the clot via a contiguous lumen formed from the working lumen of the guide sheath and the single lumen of the catheter. The system includes an instrument drive system for driving the catheter system. The instrument drive system includes a first instrument driver for driving the catheter and a second instrument driver for driving an interventional device. The system includes a remote control station for controlling the instrument drive system and the aspiration system.

In an interrelated aspect, provided is a robotic catheter system including a controller including a master input device configured for operation by a physician at a location remote from a patient; an instrument driver in communication with the controller, the instrument driver having a housing with a catheter interface movable relative to the housing, the catheter interface including a plurality of catheter drive elements coupled to respective motors located within the housing, the motors responsive to control signals generated, at least in part, by movement of the master input device for actuating the catheter drive elements and movement of the catheter interface relative to the housing. The system includes a catheter having a proximal ribbon coupled to a distal luminal portion having a single working lumen, the proximal ribbon operatively coupled to the catheter interface, the catheter axially moveable relative to a guide sheath such that movement of the catheter may be controlled by the master input device.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1A shows a patient having a catheter system inserted using a robotic drive system;

FIG. 1B shows the patient of FIG. 1A having the catheter system inserted to a level distal to a carotid siphon;

FIG. 2 is a schematic block diagram of a robotic system for a catheter procedure;

FIG. 3A is an exploded view of a catheter system;

FIG. 3B is an assembled view of the catheter system of FIG. 3A;

FIG. 3C is a detail view of a distal end region of a navigation catheter of FIG. 3A taken along circle C-C;

FIG. 4A is a schematic view of a robotic drive system having the catheter system of FIGS. 3A-3B installed;

FIG. 4B is a schematic view of a robotic drive system having the catheter system of FIGS. 3A-3B installed and nested within a larger outer diameter catheter;

FIG. 4C is a schematic view of a robotic drive system configured to provide independent movement of the guide catheter relative to other components of the catheter system;

FIG. 5 is an example of a user interface;

FIG. 6A is a side view of an implementation of a set of rollers for accommodating different outer diameters of a single component driven by the robotic drive system;

FIG. 6B is a top plan view of the rollers of FIG. 6A;

FIG. 6C is a perspective bottom view of the rollers of FIG. 6A;

FIG. 6D is a cross-sectional view of an implementation of a set of rollers as in FIG. 6A.

It should be appreciated that the drawings are for example only and are not meant to be to scale. It is to be understood that devices described herein may include features not necessarily depicted in each figure.

### DETAILED DESCRIPTION

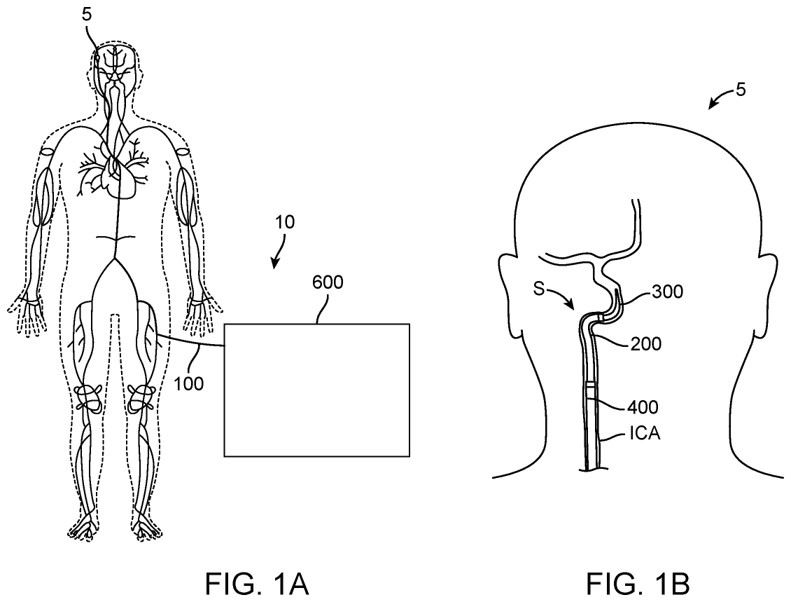

FIGS. 1A-1B illustrate a patient 5 and a robotic procedure system 10 including a distal vascular access system 100 driven by a robotic drive system 600 into the intracranial vasculature. FIG. 1B shows the distal access system 100 having a guide sheath 400 positioned within an internal carotid artery, a catheter 200 extending out a distal opening of the guide sheath 400 and a navigation catheter 300 extending out a distal opening of the catheter 200 and positioned so that at least a portion is positioned distal to a carotid siphon. The robotic procedure system 10 enables safe and rapid neurovascular access. The procedures performed by the robotic procedure system 10 can vary including diagnostic procedures as well as therapeutic procedures, such as clot removal, angioplasty, stent placement, therapy of AV malformation, aneurysm treatments, and others. The distal access system 100 is particularly useful for drawing aspiration through the catheter 200 at a location of an embolus or the delivery of an interventional device through the catheter 200 for an intracranial procedure.

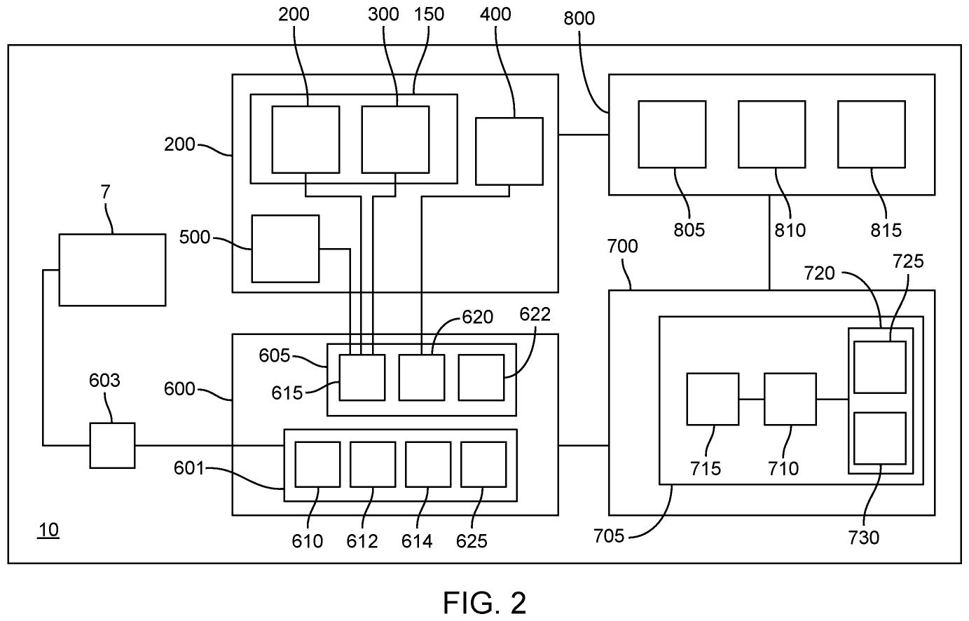

FIG. 2 is a schematic block diagram of the robotic procedure system 10. Some components of the robotic procedure system 10 are located next to the patient and other components can be remote from the patient. “Remote” as used herein may mean outside the fluoro field, but within the same room as the patient or may mean outside the same room as the patient. An operator who is remote and outside the same room as the patient need not be within the same building and can be remote from the hospital where the patient is.

The patient 5 can be supported on a table 7 that is adjacent or attached to the robotic drive system 600. The robotic drive system 600 can be controlled by an operator (e.g., doctor) at the patient's side (i.e., locally) and/or remotely using a control station 700. A control station 700 that is remote from the patient reduces radiation exposure of the operator manning the control station 700 because the operator can remain well outside the fluoro field and/or behind a radiation shield. The control station 700 also provides physical advantages to the operator in that the operator need not wear protective covering, such as lead aprons and may sit while operating the control station 700 as opposed to standing next to the operating table 7 during a procedure.

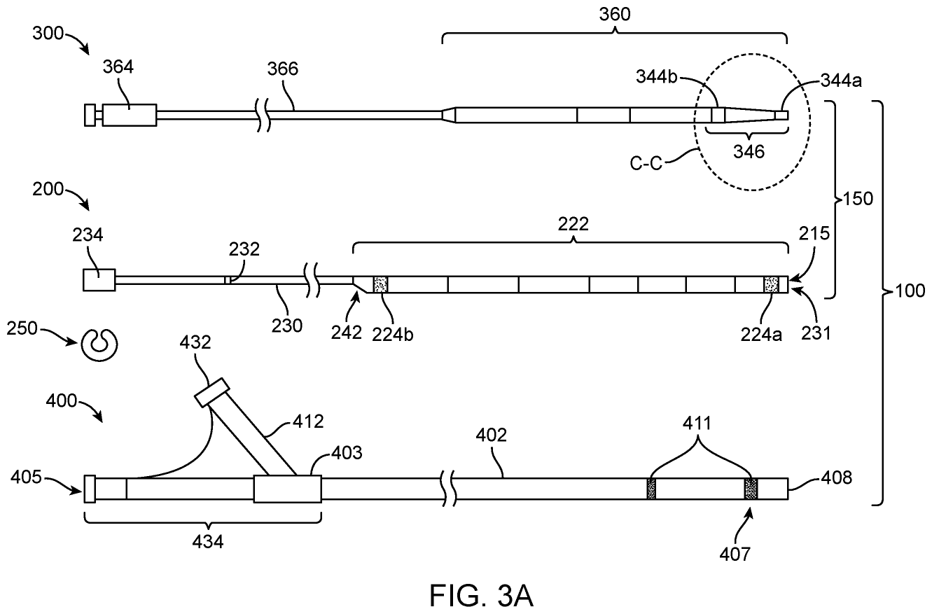

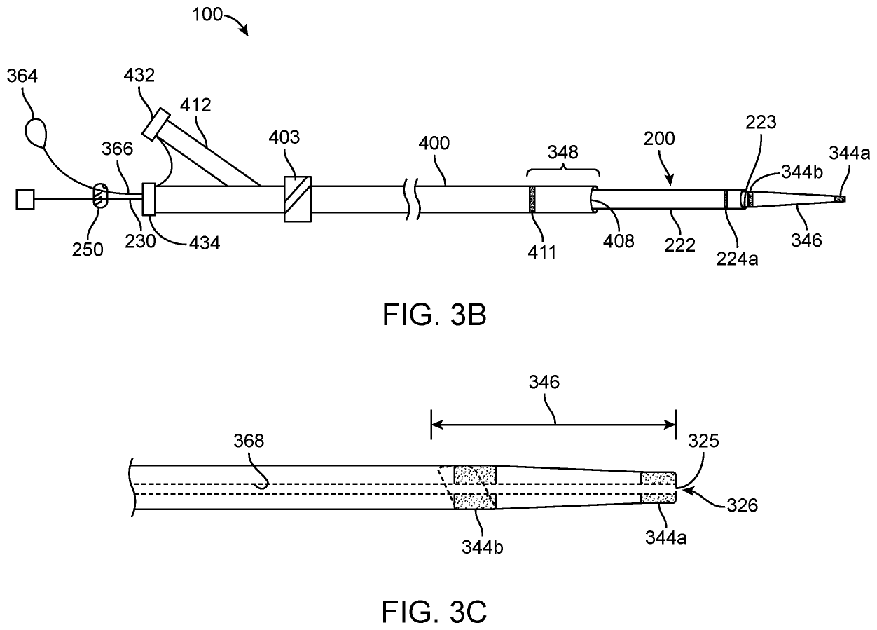

The robotic procedure system 10 can additionally include one or more other medical systems 800 including an aspiration system 805, imaging system(s) 810, contrast injection 815, and other medical systems used during a particular procedure. The other medical systems 800 can be separate systems as shown in FIG. 2 or one or more medical systems incorporated within the robotic drive system 600. For example, the robotic drive system 600 can have an integrated contrast injection, imaging, or aspiration system. The other medical systems 800 can include various patient monitoring including blood pressure, heart rate, carotid artery ultrasound, and others. Each of the various components will be described in more detail with respect to FIGS. 3A-3C, FIGS. 4A-4B, and FIG. 5.

The robotic drive system 600 can include various drive mechanisms that cause independent movement of the components of the distal access system 100 and/or other components, such as a guidewire. The movement can include advancement and retraction of the component. The movement can also include rotation of the component. The robotic drive system 600 can include a base console 601 and a cassette 605 capable of mating with the console 601. The base console 601 can include a controller 610 having a user interface with one or more inputs 612 and one or more outputs 614, and one or more sensors 625. The cassette 605 can include a plurality of roller sets 615 and a plurality of connectors 620. Use of the term “roller” is not intended to exclude other types of manipulating mechanisms. For example, the components can be advanced and/or retracted using a pad system, teeth, a screw drive, moving grips, clamps, graspers or other mechanism configured to grip, release, push, pull, twist, turn, and/or otherwise achieve feeding motion of the various components in one or more directions relative to the patient.

The base console 601 can be a durable component supported by an articulating arm 603 positioned on a support as is known in the art. The arm 603 can be locked into infinite positions relative to the patient in order to position the base console 601 and thus, the cassette 605, relative to the access site of the patient. The articulating arm 603 and associated support can be a free-standing piece of equipment movable about the operating room. The articulating arm 603 and associated support may also be configured to clamp onto another component, such as a region of the table 7 on which the patient is positioned. The base console 601 can be coupled to a region of the arm 603, such as on a rail that allows for the base console 601 to move relative to the arm 603 once coupled. Movement of the base console 601 can be manually performed by a user physically sliding the base console 601 along the rail of the arm 603. The movement of the base console 601 relative to the arm 603 can also be actuated by a user on an electronic input on the console 601 or arm 603.

The base console 601 is configured to operably couple to the cassette 605, which can be a disposable item manufactured for single use. The cassette 605 and console 601 can be coupled together at the time of use and positioned relative to a patient 5 on a table 7 using the arm 603. For example, the base console 601 can be positioned over the patient 5 adjacent a region where the access site of the catheter system into the patient is located. The coupling between the base console 601 and cassette 605 can vary. Generally, the coupling achieves both electronic and mechanical linkages between the two so that the cassette 605 can be controlled by the base console 601. The base console 601 can incorporate one or more motors and gearing that couple to drive rollers 615 within the cartridge 605. The coupling of the motors in the base console 601 to the rollers 615 in the cassette 605 may also be accomplished with magnets. For example, the base console 601 can include a motor configured to rotate a magnet in the base console 601. The base console magnet would in turn rotate a corresponding magnet in the cassette roller 615. Use of magnets provides an interface having minimal surface features that would need to be cleaned between procedures. The base console 601 can be covered by a disposable sheath 607 that is designed to be penetrated by or receive through openings formed in the disposable sheath 607 one or more connectors 620 of the cassette 605. The sheath 607 maintains a sterile environment while still allowing for coupling between the base console 601 and the cassette 605. The cassette 605 can incorporate one or more covers, windows, doors that open to allow for installation of the catheter components with the plurality of rollers 615 and connectors 620 and latch closed following insertion.

The one or more inputs 612 can be used to manipulate the arm 603 and thus, the position of the console 601 relative to the patient 5. The one or more inputs 612 can also be used to adjust the position of the console 601 (and thus, the cassette 605) relative to the arm 603, as discussed above. For example, the console 601 can couple to a rail system of the arm 603 so that the console 601 and its attached cassette 605 can be slid back and forth incrementally relative to the patient 5. In other implementations, the cassette 605 can be moved relative to the console 601 after coupling. The incremental movement can be adjusted, for example, to seat a component within an access vessel, to change an angle of the robotic drive system 600 relative to the table 7, or other adjustment.

The one or more inputs 612 can also include inputs configured to set various parameters of a procedure before control of the procedure is performed remotely. The one or more inputs 612 need not be an actual physical button and can be on a touchscreen or other sort of display device that allows for a user at the beside to initiate one or more functions of the robotic drive system 600. In some implementations, the one or more inputs 612 of the robotic drive system 600 can include a scanner that is configured to scan a 2D bar code, 3D bar code, QR code, image-based code, optically-readable code, or other code as is known in the art. For example, the cassette 605 or any of a variety of components being used on a patient may include a product label having a code configured to be scanned, the patient may have a hospital band with a code configured to be scanned. The one or more inputs 612 can be used to control the manipulation of the catheter system components including the guidewire 500, for example, by activating the rollers 615.

The robotic drive system 600 can include a sheath retainer 622 to aid in supporting, for example, the guide sheath 400 relative to the console 601 or cassette 605. The sheath retainer 622 can be a semi-rigid tube or slit sleeve capable of being advanced over at least a portion of the guide sheath 400, for example, the section of the sheath body 420 that is outside the patient that would otherwise be unsupported. The sheath retainer 622 may include a telescoping or collapsible tube section that can shorten or lengthen while providing columnar support to the body 420 of the guide sheath 400 during use. The sheath retainer 622 can prevent the body 420 from buckling when components, such as the catheters 200, 300, are advanced and retracted by the robotic drive system 600 relative to the body 420. The sheath retainer 622 can slide within a track within the cassette 605, for example, as the console 601 and cassette 605 are moved by the arm 603. The sheath retainer 622 positioned over the guide sheath 400 can be fixed to the patient at the introducer sheath at the femoral arterial access to prevent buckling of the guide sheath 400 between the site of insertion within the patient and the robot. The sheath retainer 622 supports the guide sheath 400 while still allowing the sheath 400 to be advanced and retracted by motion of the cassette 605 and/or arm 603. The sheath retainer 622 can include one or more markers that can be used to monitor the position of the sheath retainer 622 within the cassette 605 by an optical monitoring feature on the console 601 or arm 603.

One or more of the plurality of cavities and/or connectors 620 on the cassette 605 can engage the guide sheath 400 to the cassette 605. A first connector 620 can secure a proximal end of the guide sheath 400 to a connector that resides within a cavity in the cassette 605 and also secures the robotic drive system 600 to the guide sheath 400. Another cavity or connector 620 can include one or more gears configured to mate with teeth on a collar on the guide sheath 400 such that the guide sheath 400 may be rotated around its longitudinal axis A. As will be described in more detail below, the proximal end of the guide sheath 400 can include a y-connector or hub 434 with a rotating connector (referred to herein as a rotating hemostatic valve or RHV) that provides access to the working lumen of the guide sheath and also allows the guide sheath 400 to rotate. The guide sheath 400 can rotate via a gear collar mounted distal to the hub even while the y-connector stays fixed within the cassette cavity or pocket. The y-connector can be releasably secured within the cassette 605 by a connector or cavity, which can be a clamp, snap fit or other feature.

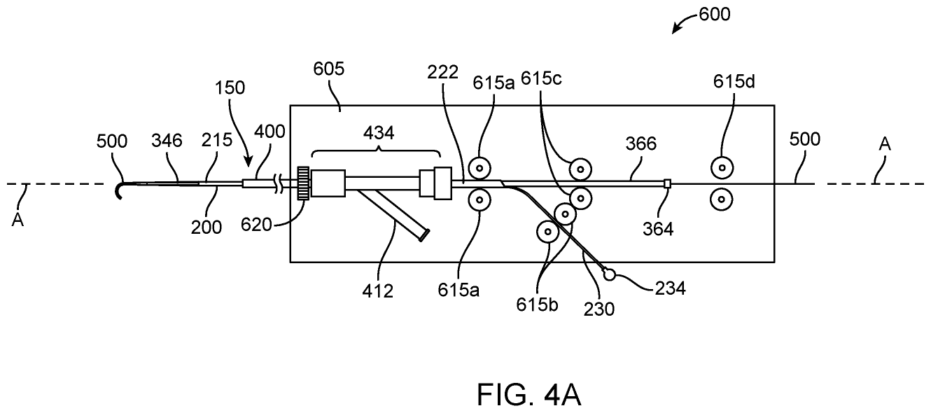

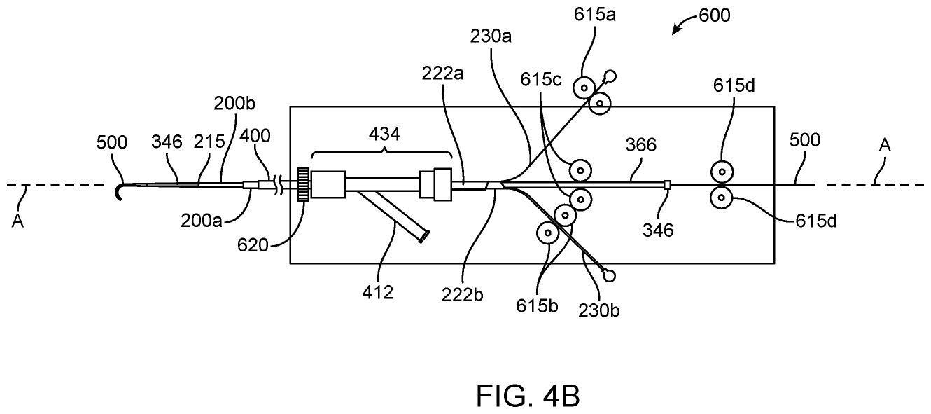

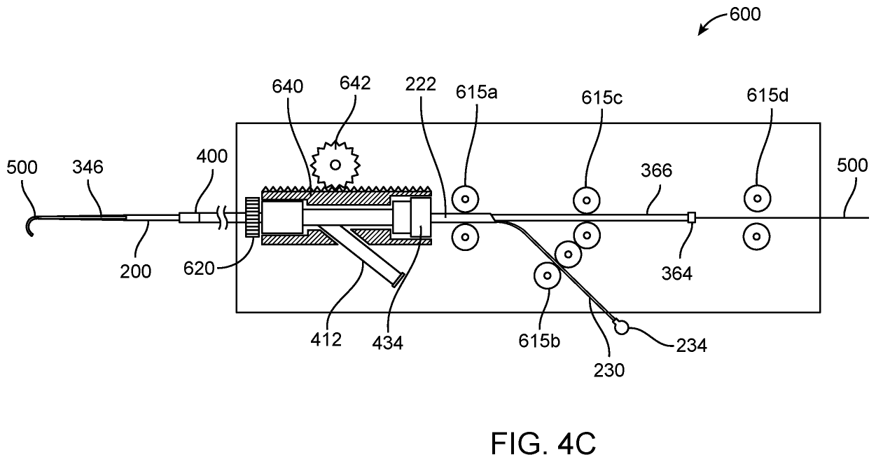

The plurality of rollers 615 can also act to engage one or more components to the cassette 605 as well as linearly translate and/or rotate the components of the distal access system 100 relative to the longitudinal axes A. The cassette 605 can have more than a single set of drive rollers 615. As will be discussed in more detail below with regard to FIGS. 4A-4B, a first set of rollers 615 can be configured to engage with the catheter 200, specifically a proximal control element 230 of the catheter 200. A second set of rollers 615 can be configured to engage with the navigation catheter 300, specifically a proximal extension 366 of the navigation catheter 300. The navigation catheter 300 extends through the lumen of the catheter 200. The second set of rollers 615 can be positioned proximal of the first set of rollers 615. The cassette 605 can include additional sets of rollers 615 located, for example, proximal of the first and second sets of rollers 615 to engage with a guidewire or another region of the one or more catheters. The sets of drive rollers 615 can include a first roller and a second roller spaced near one another to allow a component of the system to be positioned between them and to contact an external surface of the component with sufficient force to provide movement of that component upon rotation of the roller set 615. The roller set 615 can be made up of two rollers, however, as mentioned above any of a variety of conveying mechanisms are considered herein to cause advancement and retraction of the component being driven.

Again with respect to FIG. 2, the controller 610 of the robotic drive system 600 is in communication with the control station 700 configured for an operator to remotely control one or more functions of the robotic drive system 600. The motion of the catheters 200, 300 achieved by the plurality of rollers 615 can be controlled by the controller 610 of the robotic drive system 600, which is in electronic communication with the control station 700, which will be described in more detail below. The controller 610 of the robotic drive system 600 allows for the components to be directly driven at the location of the patient. For example, a technician might be stationed next to the patient and tasked with performing certain procedures by operating the robotic drive system 600 directly using one or more inputs 612.

The technician may use the controller 610 of the robotic drive system 600 initially during a first stage of a procedure on a patient and then turn to a controller 710 of the control station 700 for another part of the procedure. In a procedure, if desired by the user, the initial stage of advancement of the components of the catheter system can be performed manually by an operator until the components reaches a particular amount of advancement. The amount of manual advancement can depend on user preference as well as the procedure to be performed. For example, the tip of the guide sheath 400 can be advanced to the common carotid artery, the cervical ICA, or a more distal section of the ICA for carotid artery or anterior circulation procedures, or subclavian artery or vertebral artery for posterior circulation procedures. The catheter system including the catheter 200 and the navigation catheter 300 can be advanced manually through the guide sheath 400 a desired distance before being latched into the cassette 605, for example, up to the distal end of the guide sheath 400. A guidewire can be inserted further, for example, up to and across the target treatment site, before being latched into the cassette 605. The user can then activate or load the elements into cassette 605 so as to enable movement of the components by the drive system 600.

The controller 610 of the robotic drive system 600 may also be in communication with the control station 700, which is remote from the patient, so that a physician can cause the robotic drive system 600 to perform certain other functions via communication between the controller 710 of the control station 700 and the controller 610 of the robotic drive system 600. The controller 610 of the robotic drive system 600 can also be in communication with the one or more sensors 625 of the robotic drive system 600. The one or more sensors 625 can sense any of a variety of conditions related to the robotic drive system 600, the distal access system 100, and/or the patient 5. For example, the one or more sensors 625 can sense blood flow or pressure through one or more of the catheters.

The control station 700 can be within the same room as the robotic drive system 600, or can be physically remote from the robotic drive system 600 and the patient 5, such as within a different room. For example, the patient 5 can be within a procedure room and the control station 700 can be in a separate control room associated with the procedure room or within a room fully remote from the building within which the patient 5 is located.

The control station 700 can be a computing system 705 including, but not limited to a desktop computer, laptop computer, tablet computer, or other processing device. The computing system 705 of the control station 700 can include a controller 710, a communication port 715, and a user interface 720 including at least one input 725 and at least one display 730. The user interface 720 can include a graphical user interface (GUI).

The controller 710 of the computing system 705 can include at least one processor and a memory device. The memory device may be configured for receiving and storing user input data as well as data acquired during use of the robotic drive system 600. The memory device can include any type of memory capable of storing data and communicating that data to one or more other components of the system 10, such as the processor. The memory may be one or more of a Flash memory, SRAM, ROM, DRAM, RAM, EPROM, dynamic storage, and the like. The controller 710 of the computing system 710 can run software that initiates actions of the robotic drive system 600 with or without any user input. The controller 710 can synchronize movements of one or more of the components, such as by driving the plurality of rollers 615 at the same speed to achieve simultaneous advancement of two catheters 200, 300 as a catheter assembly. The controller 710 may run software that is programmed to detect/sense various markers on the components in order to assess relative extension and/or total distance of advancement of the components, and/or speed of advancement or withdrawal. For example, the controller 710 can determine a magnitude of linear translation of one or more of the components (e.g., catheter 200, catheter 300), such as by a marker or markers on the component being sensed by the sensors 625 of the robotic drive system 600 or by rotation of the plurality of rollers 615. For example, the linear translation of one or more of the components containing radial markers or magnets can be detected by an optical sensor or Hall sensor respectively and controlled by a programmable logic control system or software. Roller motion can be controlled by a servo motor where rotation is controlled by a programmable logic control system. The controller 710 software may be programmed to drive the plurality of rollers 615 at a certain relative speed or extension with regard to one or more components depending on where in the anatomy of the components are being advanced. The controller 710 software may include clog detection based on flow sensing through the catheters by the one or more sensors 625 of the robotic drive system 600. It should be appreciated that the controller 610 of the robotic drive system 600 and the controller 710 of the control station 700 can be integrated into a single controller for the system as a whole (i.e., only one of the controller 610 and controller 710 affect the motion of the robotic drive system and the other controller 610, 710 not affecting motion may be providing, storing, and/or displaying status, data, and the like). The controller 610 of the robotic drive system 600 and the controller 710 of the control station 700 can be different controllers that are in communication, where one controller (either 610 or 710) is the primary controller and the other controller is the secondary controller. In other words, when control information from one controller is in conflict with the second controller, the primary controller has precedence such that the two controllers 610, 710 operate according to a master/slave configuration.

One or both controllers 610 and/or 710 may display the status of components of the robotic display system. For example, the controller can display a red light if a component of the distal access system 100 is not loaded and/or latched in the associated cassette position and can switch to green when correctly loaded and/or latched. The user interface features of the system is described in more detail below.

The communication port 715 is configured to communicate with a corresponding one of the robotic drive system 600. The communication port 715 can be a wired communication port, such as a RS22 connection, USB connection, Firewire connections, proprietary connections, or any other suitable type of hard-wired connection configured to receive and/or send information to the robotic drive system 600. The communication port 715 can alternatively or additionally include a wireless communication port such that information can be fed between the control station 700 and the robotic drive system 600 via a wireless link, for example to display information in real-time on the display 730. For example, the display can show the status of loading and latching of all components of the distal access system 100 in the associated position in cassette 605. The display can additionally show a status identifier, such as a word or words (e.g., “READY”) when all components are loaded and latched. The display can also prompt the order of component loading during the loading process. The wireless connection can use any suitable wireless system, such as Bluetooth, Wi-Fi, radio frequency, ZigBee communication protocols, infrared or cellular phone systems, and can also employ coding or authentication to verify the origin of the information received. The wireless connection can also be any of a variety of proprietary wireless connection protocols.

The at least one input 725 can include a touch screen, one or more joysticks, scroll wheels, buttons, sliders, foot pedals, microphones, voice commands, and/or other inputs. The inputs 725 are configured to cause advancement, withdrawal, and/or rotation of one or more of the various components including one or more guide sheaths, catheters, microcatheters, guidewires, or other components by driving the rollers 615 of the robotic drive system 600. The inputs 725 can include an emergency button, speed control buttons or sliders, or other inputs designed to achieve a particular motion or action of the one or more components. The inputs 725 can move the plurality of rollers, such as in at least one degree of freedom. For example, the first and second sets of rollers 615 can be moved in response to an operator input so as to advance the catheters 200, 300, and/or guide sheath 400 in a distal and proximal direction.

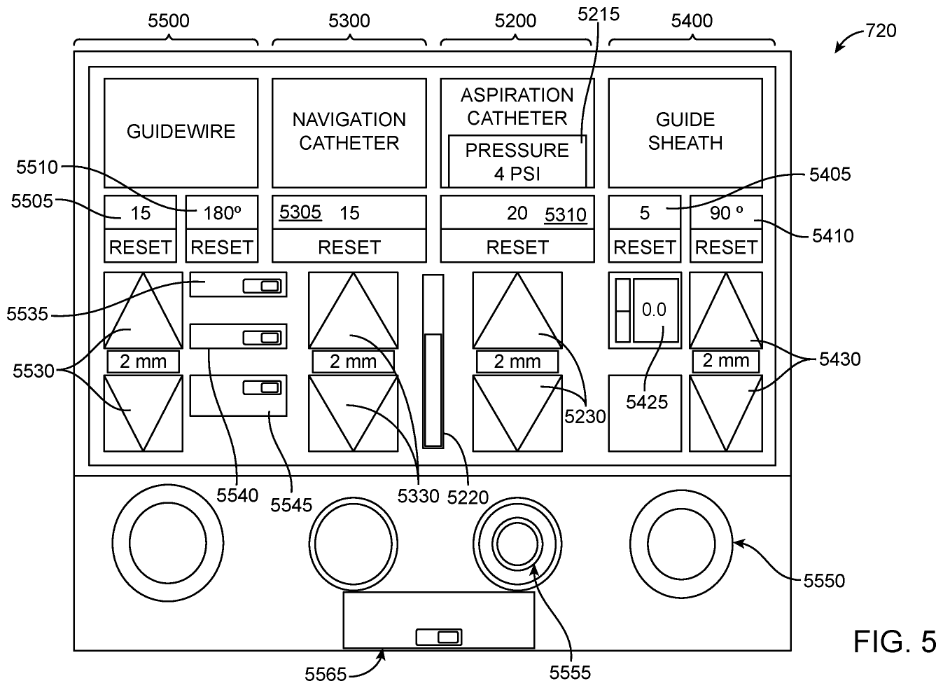

The user interface 720 can include one or more displays 730 configured to display information about the patient, the components, and/or the procedure. The user interface 720 can be a graphical user interface (GUI) (see FIG. 5). The GUI may display image data (e.g., x-ray images, MRI images, CT images, ultrasound images, etc.), patient vitals (e.g., blood pressure, heart rate, rhythm, etc.), patient history (e.g., age, weight, medical history), component data (e.g., catheter lengths, bore sizes, etc.) treatment assessment data (e.g., aspiration pressure, aspiration cycling, etc.), and any other information useful to the procedure being performed. The same information can be displayed at the patient table on an additional user interface display. This allows for technicians within the same room as the patient at the operating table to see the same information as an operator located remote to the patient and the operating table. As mentioned above, the information displayed can be coordinated with the inputs to provide better orientation to the user. These and other aspects will be described in more detail below.

Aspects of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive signals, data and instructions from, and to transmit signals, data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Each of the components of the robotic procedure system 10 will be described in more detail below, including the catheter system 100 and the robotic drive system 600.

Catheter System Components

The robotic drive system 600 of the procedure system 10 can be used to deliver one or more catheters, catheter systems, and/or devices designed to be delivered through the catheters or catheter systems. As mentioned, the procedures performed by the procedure system 10 can vary including diagnostic procedures as well as therapeutic procedures, such as clot removal, angioplasty, stent placement, therapy of AV malformation, aneurysm treatments, and others. In some implementations, the catheter systems described herein can be used for treating acute ischemic stroke (AIS). The systems described herein provide quick and simple access to distal target anatomy, in particular tortuous anatomy of the cerebral vasculature at a single point of manipulation. The rapid exchange, monopoint manipulation allows for the robotic drive system to be used with a multi-component catheter system that would otherwise be impossible due to their overall lengths creating an unsuitably large footprint within the operating room.

The medical methods, devices and systems described herein allow for navigating complex, tortuous anatomy, for example, to deliver intracranial medical devices. The extreme flexibility and deliverability of the distal access catheter systems described herein allow the catheters to take the shape of the tortuous anatomy rather than exert straightening forces creating new anatomy. The distal access catheter systems described herein can pass through tortuous loops while maintaining the natural curves of the anatomy therein decreasing the risk of vessel straightening. The distal access catheter systems described herein can thereby create a safe conduit through the neurovasculature maintaining the natural tortuosity of the anatomy for other catheters to traverse (e.g. larger bore aspiration catheters, support catheters for stent or flow diverter delivery, etc.).

While some implementations are described herein with specific regard to accessing a neurovascular anatomy for application of aspiration or the treatment of AIS, the systems and methods described herein should not be limited to this and may also be applicable to other procedures as mentioned elsewhere herein. The catheter systems described herein may be used to deliver working devices to a target vessel of a coronary anatomy or other vasculature anatomy. Where the phrase “distal access catheter” or “aspiration catheter” is used herein that the catheter can be used for aspiration, the delivery of fluids to a treatment site or as a support catheter, or distal access providing a conduit that facilitates and guides the delivery or exchange of other devices, such as a guidewire or interventional devices, such as stent retrievers, stents, flow diverters, coils, balloons, and other devices.

The devices and systems described herein are related to and can be used in combination and in the alternative with the devices and systems described in U.S. Pat. No. 10,327,790, filed Aug. 3, 2012; U.S. Pat. No. 9,561,345, filed Dec. 19, 2014; U.S. Pat. No. 9,820,761, filed Feb. 4, 2016; U.S. Publication No. 2018/0193042, filed on Jan. 9, 2018; U.S. Publication No. 2018/0361114, filed on Jan. 19, 2018; U.S. Publication No. 2019/0351182, filed May 16, 2019; U.S. Pat. No. 11,400,255, filed Nov. 14, 2019; and U.S. Publication No. 2020/0289136, filed Jun. 2, 2020. The disclosures of each of these publications and applications are incorporated by reference herein in their entireties.

FIGS. 3A-3B illustrate an implementation of a distal access system 100 including a catheter assembly 150 configured to be advanced using the robotic drive system 600. The catheter assembly 150 can include a first catheter 200 and a second catheter 300 configured to be positioned inside the first catheter 200. The first catheter 200 may be referred to herein as a distal catheter or an outer catheter. The second catheter 300 may be referred to herein as a navigation catheter or an inner catheter. FIG. 3A is an exploded view of the system 100 and FIG. 3B is an assembled view of the system 100 of FIG. 3A. FIG. 3C is a detailed view of the navigation catheter 300 of the catheter assembly 150 of FIG. 3A taken along circle C-C. The system 100 is capable of providing quick and simple access to distal target anatomy, particularly the tortuous anatomy of the cerebral vasculature. As will be described in more detail below, all wire and catheter manipulations can occur at or in close proximity to a single rotating hemostatic valve (RHV) or more than a single RHV co-located in the same device. The manipulations can occur as a result of the robotic drive system 600 and also via manual manipulations by an operator at the patient's side. The procedures described herein can involve a combination of both manual operator manipulations as well as robotic drive system manipulations.

The system 100 can include one or more catheter assemblies 150, each having the first catheter 200 and the inner, navigation catheter 300. The first catheter 200 can be an aspiration catheter designed to seal with the catheter through which it extends or an access catheter designed without a seal. The first catheter 200 need not seal and can be used as a support catheter for another component. The catheter assembly 150 is configured to be advanced through an access guide sheath 400. The catheter 200 is configured to be received through the guide sheath 400 and is designed to have exceptional deliverability. The catheter 200 can be a spined catheter that when assembled within a guide sheath provides a step-up in inner diameter with a lumen of the guide sheath 400. The catheter 200 can be delivered using the navigation catheter 300 inserted through a lumen 223 of the catheter 200. The flexibility and deliverability of the first catheter 200 allow the catheter 200 to take the shape of the tortuous anatomy as it is advanced and avoids exerting straightening forces on the anatomy. The first catheter 200 is capable of this even in the presence of the navigation catheter 300 extending through its lumen. Thus, the flexibility and deliverability of the navigation catheter 300 is on par or better than the flexibility and deliverability of the distal luminal portion 222 of the first catheter 200 in that both are configured to reach the middle cerebral artery (MCA) circulation without straightening out the curves of the anatomy along the way.

The system 100 can be a distal access system that can create a variable length from point of entry at the percutaneous arteriotomy (e.g., the femoral artery or other point of entry) to the target control point of the distal catheter. Conventional distal access systems for neurointerventions typically include a long guide sheath or guide catheter placed through a shorter “introducer” sheath (e.g., 11-30 cm in length) at the groin. The long guide sheath is typically positioned in the ICA to support neurovascular interventions including stroke embolectomy (sometimes referred to as “thrombectomy”). For added support, these can be advanced up to the bony terminal petrous and rarely into the cavernous or clinoid or supraclinoid terminal ICA when possible. To reach targets in the M1 or M2 distribution with devices for mechanical thrombectomy, such as devices for manual aspiration thrombectomy (MAT), stent retriever (SR), aspiration first pass technique (ADAPT), and “Solumbra” (Aspiration+SR), an additional catheter may be inserted through the long guide catheter. These catheters are typically large-bore aspiration catheters that can be, for example 130 cm in length or longer. As will be described in more detail below, the distal access systems 100 described herein can be shorter, for example, only 115 cm in length when taken as a system as measured from the access point, typically the common femoral artery. Additionally, the systems described herein can be inserted through a single rotating hemostatic valve (RHV) 434 on the guide sheath 400 or more than one RHV co-located in the same device, such as a dual-headed RHV.

Still with respect to FIGS. 3A-3B, the distal access system 100 can include an access guide sheath 400 having a body 402 through which a working lumen extends from a proximal hemostasis valve 434 coupled to a proximal end region 403 of the body 402 to a distal opening 408 of a distal end region. The working lumen is configured to receive the catheter 200 therethrough such that a distal end of the catheter 200 can extend beyond a distal end of the sheath 400 through the distal opening 408. The guide sheath 400 can be used to deliver the catheters described herein as well as any of a variety of working devices known in the art. For example, the working devices can be configured to provide thrombotic treatments and can include large-bore catheters, aspiration embolectomy (sometimes referred to as thrombectomy), advanced catheters, wires, balloons, retrievable structures, such as coil-tipped retrievable stents “stent retriever”, stents, and flow diverters.

The sheath body 402 can extend from a proximal furcation or rotating hemostatic valve (RHV) 434 at the proximal end region 403 to a distal end region 407 of the body 402. The proximal RHV 434 may include one or more lumens molded into a connector body to connect to the working lumen 405 of the body 402 of the guide sheath 400. The working lumen can receive the catheter 200 and/or any of a variety of working devices for delivery to a target anatomy. The RHV 434 can be constructed of thick-walled polymer tubing or reinforced polymer tubing. The RHV 434 allows for the introduction of devices through the guide sheath 400 into the vasculature, while preventing or minimizing blood loss and preventing air introduction into the guide sheath 400. The RHV 434 can be integral to the guide sheath 400 or the guide sheath 400 can terminate on a proximal end in a female Luer adaptor to which a separate hemostasis valve component, such as a passive seal valve, a Tuohy-Borst valve or RHV may be attached. The RHV 434 can have an adjustable opening that is open large enough to allow removal of devices that have adherent clot on the distal opening 408 without causing the clot to dislodge at the RHV 434 during removal. Alternately, the RHV 434 can be removable, such as when a device is being removed from the sheath 400 to prevent clot dislodgement at the RHV 434. The RHV 434 can be a dual RHV or a multi-head RHV.

The RHV 434 can form a Y-connector on the proximal end region 403 of the sheath 400 such that the first port of the RHV 434 can be used for insertion of a working catheter into the working lumen of the sheath 400 and a second port into arm 412 can be used for another purpose. For example, a syringe or other device can be connected at arm 412 via a connector 432 to deliver a forward drip, a flush line for contrast agent or saline injections through the body 402 with or without a catheter toward the distal opening 408 and into the target anatomy. Arm 412 can also connect to a vacuum source. The vacuum source can be an active source of aspiration, such as an aspiration pump, a regular or locking syringe, a hand-held aspirator, hospital suction, or the like, configured to draw suction through the working lumen. In an embodiment, the vacuum source is a locking syringe (for example a VacLok Syringe) attached to a flow controller. The operator can pull the plunger on the syringe back into a locked position while the connection to the flow line is closed prior to an embolectomy step of a procedure. In another implementation, the arm 412 can be connected to a vacuum source that is a pump configured to apply a constant or variable aspiration pressure through the working lumen of the guide sheath 400. The single, shared source of aspiration is sufficient to draw aspiration through the entire system 100, even when multiple aspiration catheters 200 are nested within one another through the working lumen of the guide sheath 400. The arm 412 can also allow the guide sheath 400 to be flushed with saline or radiopaque contrast agent during a procedure. The working lumen can extend from the distal opening 408 to a working proximal port of the proximal end region 403 of the sheath body 402.

Contrast agent (e.g., from the contrast injection system 815) can be injected through the guide sheath 400 into the vessel to visualize an occlusion site by angiogram. For example, the guide sheath 400 can be positioned so that at least a portion is positioned within the carotid artery. The contrast agent may be injected through the sheath 400 once positioned in this location. Contrast agent can also be injected through one or more catheters inserted through the guide sheath 400. A baseline angiogram can be obtained, for example in the anterior/posterior (AP) and/or lateral views, prior to device insertion to assess occlusion location by injection of contrast media through the sheath 400 with fluoroscopic visualization (e.g., part of the imaging system 810). Fluoroscopic visualization may continue as the catheter system is advanced and subsequent angiograms can be captured periodically and particularly after every attempt to retrieve the embolus to assess reperfusion. The baseline angiogram image can be superimposed, such as with digital subtraction angiography, so that the vasculature and/or occlusion site are visible while the catheter system is advanced. Once the catheter assembly 150 is advanced into position (the positioning will be described in more detail below), the navigation catheter 300 can be withdrawn and removed from the system. In some implementations, such as for the treatment of an occlusion by aspiration embolectomy, a vacuum source, such as a pump, may be connected to the sheath 400 and activated to direct aspiration to the distal end of the catheter 200. The aspiration may be applied for a period of time (e.g., between about 30 seconds up to about 3 minutes, preferably about 2 minutes) to allow for capture and engulfment of the embolus in the catheter 200. The flow rate of aspiration may vary and in one example can be between about 25 inches Hg (inHg) (12.279 psi) up to about 28 inHg (13.752 psi). In some implementations, the pump is allowed to run to build up a vacuum outside of the patient over a first period prior to applying the vacuum to the vessel, for example, by turning a flow control switch to an “on” position. In other implementations, the pump is turned on at a particular flow rate and is applied to the vessel immediately allowing for the build-up of vacuum through the system. After applying aspiration to the catheter for a period of time, the catheter 200 can be slowly withdrawn. Once free flow is achieved, observable by continuous collection of fluid within a receptacle, the aspiration source can be disconnected from the sheath 400 and a confirmatory angiogram performed. The angiogram can be performed by injecting contrast agent through the aspiration catheter 200 still positioned through the working lumen of the sheath 400. The angiogram can also be performed through the guide sheath 400 after complete removal of the aspiration catheter 200 from the guide sheath 400.

The aspiration can be performed by the aspiration system 805, the contrast injected by the contrast injection system 815, and the angiogram performed by the imaging system 810 each under the control of the control station 700 (see FIG. 2).

The vacuum source can increase in aspiration level when the flow rate is slow and decrease when the flow rate is increased. In this manner, the force is greatest when the catheter is clogged or partially clogged, but decreases to a minimal level when there is free flow to ensure protection from distal emboli but limit the volume of aspirated blood. In this manner, the system can optimize the embolus aspiration while limiting the amount of blood aspirated. When the flow in the catheter 200 is blocked or restricted, the pump can create a higher level of vacuum. In this example, the aspiration force may be configured to rise when higher vacuum is detected, such as by the one or more sensors 625 (see FIG. 2). The vacuum source can include a vacuum gauge or the vacuum gauge may be incorporated into the RHV or the Luer or proximal end of the guide sheath 400. In still further implementations, the robotic drive system 600 can incorporate one or more sensors 625 configured to assess the status of one or more functions of the system, such as flow rate, clogging, level of vacuum, etc. to automatically adjust the parameters of the system. The various controls of the systems described herein can be performed by an operator at the control station 700 or manually at the operating table 7 or a combination thereof.

In an implementation, the guide sheath 400 includes one or more radiopaque markers 411. The radiopaque markers 411 can be disposed near the distal opening 408. For example, a pair of radiopaque bands may be provided. The radiopaque markers 411 or markers of any of the system components can be swaged, painted, embedded, or otherwise disposed in or on the body. In some implementations, the radiopaque markers include a barium polymer, tungsten polymer blend, tungsten-filled or platinum-filled marker that maintains flexibility of the devices and improves transition along the length of the component and its resistance to kinking. In some implementations, the radiopaque markers are a tungsten-loaded PEBAX or polyurethane that is heat welded to the component.

The guide sheath markers 411 are shown in the figures as rings around a circumference of one or more regions of the body 402. However, the markers 411 can have other shapes or create a variety of patterns that provide orientation to an operator regarding the position of the distal opening 408 within the vessel. Accordingly, an operator may visualize a location of the distal opening 408 under fluoroscopy (which can be displayed to the operator on a display 730 of the control system 700) to confirm that the distal opening 408 is directed toward a target anatomy where a catheter 200 is to be delivered. For example, radiopaque marker(s) 411 allow for rotation of the body 402 of the guide sheath 400 at an anatomical access point, e.g., a groin of a patient, such that the distal opening provides access to an ICA by subsequent working device(s), e.g., catheters and wires advanced to the ICA. In some implementations, the radiopaque marker(s) 411 include platinum, gold, tantalum, tungsten or any other substance visible under an x-ray fluoroscope. Any of the various components of the systems described herein can incorporate radiopaque markers.

Still with respect to FIGS. 3A-3B, the catheter 200 can include a relatively flexible, distal luminal portion 222 coupled to a stiffer, kink-resistant proximal extension or proximal control element 230. The term “control element” as used herein can refer to a proximal region configured for pushing movement in a distal direction as well as pulling movement in a proximal direction. The control element can be pushed and pulled manually by an operator or by the robotic drive system 600 as described elsewhere herein. The control elements described herein may also be referred to as spines, tethers, push wires, push tubes, or other elements having any of a variety of configurations. The proximal control element 230 can be a hollow or tubular element. The proximal control element 230 can also be solid and have no inner lumen, such as a solid rod, ribbon or other solid wire type element coupled on a proximal end to a tab 234 or other sort of component. Generally, the proximal control elements described herein are configured to move its respective component (to which it may be attached or integral) in a bidirectional manner through a lumen.

A single, inner lumen 223 extends through the luminal portion 222 between a proximal end and a distal end of the luminal portion 222. In some implementations, a proximal opening 242 into the lumen 223 can be located near where the proximal control element 230 coupled with the distal luminal portion 222. In other implementations, the proximal opening 242 into the lumen 223 is at a proximal end region of the catheter 200. A distal opening 231 from the lumen 223 can be located near or at the distal-most end 215 of the luminal portion 222. The inner lumen 223 of the catheter 200 can have a first inner diameter and the working lumen 405 of the guide sheath 400 can have a second, larger inner diameter. Upon insertion of the catheter 200 through the working lumen 405 of the sheath 400, the lumen 223 of the catheter 200 can be configured to be fluidly connected and contiguous with the working lumen of the sheath 400 such that fluid flow into and/or out of the system 100 is possible, such as by applying suction from a vacuum source coupled to the system 100 at a proximal end. The combination of sheath 400 and catheter 200 can be continuously in communication with the bloodstream during aspiration at the proximal end with advancement and withdrawal of catheter 200.

The distal luminal portion 222 of the catheter 200 can have a plurality of radiopaque markings 224. A first radiopaque marker 224a can be located near the distal-most end 215 to aid in navigation and proper positioning of the distal-most end 215 under fluoroscopy. Additionally, a proximal region of the catheter 200 may have one or more proximal radiopaque markers 224b so that the overlap region 348 can be visualized as the relationship between a radiopaque marker 411 on the guide sheath 400 and the radiopaque marker 224b on the catheter 200. The proximal region of the catheter 200 may also have one or more radiopaque markings providing visualization, for example, near the proximal opening 242 into the single lumen 223 of the catheter 200 as will be described in more detail below. In an implementation, the two radiopaque markers (marker 224a near the distal-most end 215 and a more proximal marker 224b) are distinct to minimize confusion of the fluoroscopic image, for example the catheter proximal marker 224b may be a single band and the marker 411 on the guide sheath 400 may be a double band and any markers on a working device delivered through the distal access system can have another type of band or mark. The radiopaque markers 224 of the distal luminal portion 222, particularly those near the distal end region navigating extremely tortuous anatomy, can be relatively flexible such that they do not affect the overall flexibility of the distal luminal portion 222 near the distal end region. The radiopaque markers 224 can be tungsten-loaded or platinum-loaded markers that are relatively flexible compared to other types of radiopaque markers used in devices where flexibility is not paramount. In some implementations, the radiopaque marker can be a band of tungsten-loaded PEBAX having a durometer of Shore 35D.

The proximal control element 230 can include one or more markers 232 to indicate the overlap between the distal luminal portion 222 of the catheter 200 and the sheath body 402 as well as the overlap between the distal luminal portion 222 of the catheter 200 and other interventional devices that may extend through the distal luminal portion 222. At least a first mark can be an RHV proximity marker positioned so that when the mark is aligned with the sheath proximal hemostasis valve 434 during insertion of the catheter 200 through the guide sheath 400, the catheter 200 is positioned at the distal-most position with the minimal overlap length needed to create the seal between the catheter 200 and the working lumen. At least a second mark 232 can be a Fluoro-saver marker that can be positioned on the control element 230 and located a distance away from the distal-most end 215 of the distal luminal portion 222. In some implementations, a mark 232 can be positioned about 100 cm away from the distal-most end 215 of the distal luminal portion 222.

The various markers can be sensed by the one or more sensors 625 of the robotic drive system 600 to provide information regarding relative extension of the various components as well as location of the components within the anatomy based on total extension achieved.

Although the navigation catheter 300 is described herein in reference to catheter 200 it can be used to advance other catheters and it is not intended to be limiting to its use. For example, the navigation catheter 300 can be used to deliver a 5MAX Reperfusion Catheter (Penumbra, Inc., Alameda, CA), REACT aspiration catheter (Medtronic), or Sophia Plus aspiration catheter (Terumo) for clot removal in patients with acute ischemic stroke or other reperfusion catheters known in the art. Thus, where the catheter is described as having a proximal control element, the catheter can be a full length catheter and the proximal control element 230 may refer simply to a proximal tubular portion of the catheter.

Still with respect to FIGS. 3A-3B and also FIG. 3C, the navigation catheter 300 can include a non-expandable, flexible elongate body 360 coupled to a proximal extension 366. Like the catheter 200, the navigation catheter 300 may be configured for rapid exchange methods. The flexible elongate body 360 can also be configured for over-the-wire methods and be a tubular portion having a guidewire lumen 368 extending the entire length of the navigation catheter 300. In this implementation, the navigation catheter 300 can have a proximal opening from the lumen 368 that is configured to remain outside the patient's body during use. Alternatively, the tubular portion can have a proximal opening positioned such that the proximal opening remains inside the patient's body during use. The lumen 368 for the guidewire can extend a limited distance from the distal opening of the navigation catheter (e.g., 5 cm-25 cm) to the proximal opening. The proximal extension 366 can be a proximal element coupled to a distal tubular portion 360 and extending proximally therefrom. A proximal opening from the tubular portion 360 can be positioned near where the proximal element 366 couples to the tubular portion 360. Alternatively, the proximal extension 366 can be a proximal extension of the tubular portion 360 having a length that extends to a proximal opening near a proximal terminus of the navigation catheter 300 (i.e., outside a patient's body). A luer 364 can be coupled to the proximal extension 366 at the proximal end region so that tools, such as a guidewire, can be advanced through the lumen 368 of the navigation catheter 300. A syringe or other component can be coupled to the luer 364 in order to draw a vacuum and/or inject fluids through the lumen 368. The syringe coupled to the luer 364 can also be used to close off the lumen of the navigation catheter 300 to maximize the piston effect described elsewhere herein.

The configuration of the proximal extension 366 of the navigation catheter 300 can vary. In some implementations, the proximal extension 366 is simply a proximal extension of the flexible elongate body 360 that does not change significantly in structure but changes significantly in flexibility. For example, the proximal extension 366 transitions from the very flexible distal regions of the navigation catheter 300 towards less flexible proximal regions of the navigation catheter 300. The proximal extension 366 provides a relatively stiff proximal end suitable for manipulating and torqueing the more distal regions of the navigation catheter 300. In other implementations, the proximal extension 366 is a hypotube. The hypotube may be exposed or may be coated by a polymer. In still further implementations, the proximal extension 366 may be a tubular polymer portion reinforced by a coiled ribbon or braid. The proximal extension 366 can have the same outer diameter as the flexible elongate body or can have a smaller outer diameter as the flexible elongate body.

The proximal extension 366 need not include a lumen. For example, the proximal extension 366 can be a solid rod, ribbon, or wire have no lumen extending through it that couples to the tubular elongate body 360. Where the proximal extension 366 is described herein as having a lumen, it should be appreciated that the proximal extension 366 can also be solid and have no lumen. The proximal extension 366 is generally less flexible than the elongate body 360 and can transition to be even more stiff towards the proximal-most end of the proximal extension 366. Thus, the navigation catheter 300 can have an extremely soft and flexible distal end region 346 that transitions proximally to a stiff proximal extension 366 well suited for pushing and/or torqueing the distal elongate body 360.

The elongate body 360 can be received within and extended through the internal lumen 223 of the distal luminal portion 222 of the catheter 200 (see FIG. 3B). The elongate body 360 or tubular portion can have an outer diameter. The tubular portion of the navigation catheter 300 can have an outer diameter that has at least one snug point. A difference between the inner diameter of the catheter 200 (e.g., the inner diameter of the lumen at the distal end of the distal catheter portion) and the outer diameter of the tubular portion at the snug point can be no more than about 0.015″ (0.381 mm), or can be no more than about 0.010″ (0.254 mm), for example, from about 0.003″ (0.0762 mm) up to about 0.012″ (0.3048 mm), preferably about 0.005″ (0.127 mm) to about 0.010″ (0.254 mm), and more preferably about 0.007″ (0.1778 mm) to about 0.009″ (0.2286 mm). The at least one snug point of this tubular portion can be a point along the length of the tubular portion. The at least one snug point of this tubular portion can have a length that is at least about 5 cm up to about 50 cm, including for example, at least about 6 cm, at least about 7 cm, at least about 8 cm, at least about 9 cm, at least about 10 cm, at least about 11 cm, or at least about 12 cm up to about 50 cm. This length need not be uniform such that the length need not be snug along its entire length. For example, the snug point region can include ridges, grooves, slits, or other surface features.

As will be described in more detail below, the navigation catheter 300 can also include a distal end region 346 located distal to the at least one snug point of the tubular portion. The distal end region 346 can have a length and taper along at least a portion of the length. The distal end region 346 of the navigation catheter 300 can be extended beyond the distal end of the catheter 200 as shown in FIG. 3B. The proximal extension 366 of the navigation catheter 300 or proximal extension is coupled to a proximal end region of the elongate body 360 and extends proximally therefrom. The proximal extension 366 can be less flexible than the elongate body 360 and configured for bi-directional movement of the elongate body 360 of the navigation catheter 300 within the luminal portion 222 of the catheter 200, as well as for movement of the catheter assembly 150 as a whole. The elongate body 360 can be inserted in a coaxial fashion through the internal lumen 223 of the luminal portion 222. The outer diameter of at least a region of the elongate body 360 can be sized to substantially fill at least a portion of the internal lumen 223 of the luminal portion 222.

The overall length of the navigation catheter 300 (e.g. between the proximal end through to the distal-most end 325) can vary, but generally is long enough to extend through the support catheter 200 plus at least a distance beyond the distal end of the support catheter 200 while at least a length of the proximal extension 366 remains outside the proximal end of the guide sheath 400 and outside the body of the patient. In some implementations, the overall length of the navigation catheter 300 is about 145 to about 150 cm and has a working length of about 140 cm to about 145 cm from a proximal tab or hub to the distal-most end 325. The elongate body 360 can have a length that is at least as long as the luminal portion 222 of the catheter 200 although the elongate body 360 can be shorter than the luminal portion 222 so long as at least a minimum length remains inside the luminal portion 222 when a distal portion of the elongate body 360 is extended distal to the distal end of the luminal portion 222 to form the snug point or snug region with the catheter. In some implementations, this minimum length of the elongate body 360 that remains inside the luminal portion 222 when the distal end region 346 is positioned at its optimal advancement configuration is at least about 5 cm, at least about 6 cm, at least about 7 cm, at least about 8 cm, at least about 9 cm, at least about 10 cm, at least about 11 cm, or at least about 12 cm up to about 50 cm. In some implementations, the shaft length of the distal luminal portion 222 can be about 35 cm up to about 75 cm and shorter than a working length of the guide sheath and the insert length of the elongate body 360 can be at least about 45 cm, 46 cm, 47 cm, 48 cm, 48.5 cm, 49 cm, 49.5 cm up to about 85 cm.

The length of the elongate body 360 can allow for the distal end of the elongate body 360 to reach cerebrovascular targets or occlusions within, for example, segments of the internal carotid artery including the cervical (C1), petrous (C2), lacerum (C3), cavernous (C4), clinoid (C5), ophthalmic (C6), and communicating (C7) segments of the internal carotid artery (ICA) as well as branches off these segments including the M1 or M2 segments of the middle cerebral artery (MCA), anterior cerebral artery (ACA), anterior temporal branch (ATB), and/or posterior cerebral artery (PCA). The distal end region of the elongate body 360 can reach these distal target locations while the proximal end region of the elongate body 360 remains proximal to or below the level of severe turns along the path of insertion. For example, the entry location of the catheter system can be in the femoral artery and the target occlusion location can be distal to the right common carotid artery, such as within the M1 segment of the middle cerebral artery on the right side. The proximal end region of the elongate body 360 where it transitions to the proximal extension 366 can remain within a vessel that is proximal to severely tortuous anatomy, such as the carotid siphon, the right common carotid artery, the brachiocephalic trunk, the take-off into the brachiocephalic artery from the aortic arch, the aortic arch as it transitions from the descending aorta. This avoids inserting the stiffer proximal extension 366, or the material transition between the stiffer proximal extension 366 and the elongate body 360, from taking the turn of the aortic arch or the turn of the brachiocephalic take-off from the aortic arch, which both can be very severe. The lengths described herein for the distal luminal portion 222 also can apply to the elongate body 360 of the navigation catheter.

The proximal extension 366 can have a length that varies as well. In some implementations, the proximal extension 366 is about 90 cm up to about 95 cm. The distal portion extending distal to the distal end of the luminal portion 222 can include distal end region 346 that protrudes a length beyond the distal end of the luminal portion 222 during use of the navigation catheter 300. The distal end region 346 of the elongate body 360 that is configured to protrude distally from the distal end of the luminal portion 222 during advancement of the catheter 200 through the tortuous anatomy of the cerebral vessels, as will be described in more detail below. The proximal extension 366 coupled to and extending proximally from the elongate body 360 can align generally side-by-side with the proximal control element 230 of the catheter 200. The arrangement between the elongate body 360 and the luminal portion 222 can be maintained during advancement of the catheter 200 through the tortuous anatomy to reach the target location for treatment in the distal vessels and aids in preventing the distal end of the catheter 200 from catching on tortuous branching vessels, as will be described in more detail below.

In some implementations, the elongate body 360 can have a region of relatively uniform outer diameter extending along at least a portion of its length and the distal end region 346 tapers down from the uniform outer diameter. The outer diameter of the elongate body 360 can include a step-down at a location along its length, for example, a step-down in outer diameter at a proximal end region where the elongate body 360 couples to the proximal extension 366. Depending upon the inner diameter of the catheter 200, the difference between the inner diameter of catheter 200 and the outer diameter of the elongate body 360 along at least a portion of its length, such as at least 10 cm of its length, preferably at least 15 cm of its length can be no more than about 0.015″ (0.381 mm), such as within a range of about 0.003″-0.015″ (0.0762 mm-0.381 mm) or between 0.006″-0.010″ (0.1524 mm-0.254 mm). Thus, the clearance between the catheter 200 and the elongate body 360 can result in a space on opposite sides that is no more than about 0.008″ (0.2032 mm), or can be no more than about 0.005″ (0.127 mm), for example, from about 0.001″ up to about 0.006″ (0.0254 mm-0.1524 mm), preferably about to about 0.005″ (0.0508 mm-0.127 mm), and more preferably about 0.003″ to about (0.0762 mm-0.0508 mm).

The navigation catheter 300 has a large outer diameter and a relatively small inner diameter, particularly when a guidewire extends into or through the lumen of the navigation catheter 300. The lumen of the navigation catheter 300 substantially filled by the guidewire and/or liquid creates a closed system with the catheter 200. The navigation catheter 300 substantially fills or is substantially occlusive to the catheter 200 creating a piston arrangement within the catheter lumen. Withdrawing the occlusive navigation catheter 300 through the catheter lumen creates an internal vacuum within the catheter 200 like a plunger in a syringe barrel. The internal vacuum created within the distal end region of the catheter 200 can draw embolic material towards and/or through the distal end 215 of the catheter 200 positioned at or near the face of the embolus. As mentioned above, a syringe or flush can be coupled to the luer 364 prior to withdrawal of the navigation catheter 300 from the catheter lumen. The syringe coupled to the luer 364 of the navigation catheter 300 closes the system and maximizes the piston effect upon withdrawal. The internal vacuum can begin to clear clot material proximal to the embolus or draw the embolus itself into the distal end of the catheter even before external aspiration is applied at the proximal RHV of the base sheath. Further, the catheter system as it is advanced through the tortuous neuroanatomy can store energy or forces, for example, in the compression of the catheter 200 before the navigation catheter 300 is withdrawn. The extreme tortuosity of the intracerebral vasculature, particularly around the bony structures of the skull can require more severe force to traverse in combination with the dramatic transition in the size between vessels to reach the occlusion site, such as the large aorta and 1-3 mm sized target vessel, can cause stored forces or energy in a catheter. Withdrawal of the navigation catheter 300 can release this stored energy causing distally-directed movement of the distal catheter portion 222. The distally-directed movement of the distal catheter portion 222 can be exploited, for example, when treating an embolus by aspiration thrombectomy. The distal catheter portion 222 may be allowed to move (i.e., by an operator and/or the robotic system 600 releasing the grip on the catheter) towards the embolus to atraumatically nest, seat, and/or embed the distal end 215 of the catheter 200 with the proximal face of the embolus for optimum positioning of the catheter 200 relative to the embolus. Withdrawing the navigation catheter 300 through the catheter lumen can achieve a more successful one-pass embolectomy by creating an initial internal vacuum within the distal end region of the catheter 200 alone or in combination with the distally-directed movements of the distal catheter portion 222.

Various movements of the catheter 200 and/or the navigation catheter 300 during use of the system, for example withdrawal of the navigation catheter to achieve the piston effect, can be performed manually by the operator located at the operating table 7 or by the robotic drive system 600, for example, controlled by an operator at the control station 700. The drive system 600 can be programmed to withdraw the navigation catheter 300 from the catheter lumen at a selected velocity. The selected velocity can vary anywhere from about 4 cm per second withdrawal up to about 165 cm per second withdrawal. In some implementations, the withdrawal velocity is selected so that the piston arrangement creates an aspiration pressure at the distal end region of the catheter 200 that remains in position at the occlusion. As an example, the catheter system 150 can be assembled so that the tapered distal end region 346 of the navigation catheter 300 extends distal to the distal end of the catheter 200. The navigation catheter outer dimensions can substantially fill at least a portion of the lumen of the catheter 200 creating a piston arrangement. The length to create the piston arrangement can be at least about 10 cm of the catheter length. The length can be about 4 mm to about 75 cm. The assembled catheter system can be advanced together (either by a clip coupling the two components or by two roller sets synchronized to advance them simultaneously) towards an occlusion site in a cerebral vessel. A syringe may be coupled to the luer 364 of the navigation catheter 300 prior to its withdrawal from the catheter lumen. The syringe closes the lumen and thus, the system 150 thereby maximizing the piston effect upon withdrawal of the navigation catheter 300. The robotic drive system 600 can then be used to withdraw the navigation catheter 300 at the selected velocity (e.g., from 20-25 cm per second up to about 160 cm per second).

The elongate body 360 can have an overall shape profile from proximal end to distal end that transitions from a first outer diameter having a first length to a tapering outer diameter having a second length. The first length of this first outer diameter region (i.e., the snug-fitting region between the distal luminal portion 222 and the elongate body 360) can be at least about 5 cm, or 10 cm, up to about 50 cm. In other implementations, the snug-fitting region can extend from the proximal tab or luer 364 substantially to the tapered distal end region 346 which depending on the length of the navigation catheter 300, can be up to about 170 cm.

In some implementations, the length of the tapering outer diameter of the distal end region 346 can be between 1 cm and 4 cm. In other implementations, the length of the tapering outer diameter can be over a length of 0.5 cm to 5 cm. In still other implementations, the length of the tapering outer diameter can be over a length of 2 cm to 3 cm. The distal end region 346 of the elongate body 360 can also be shaped with or without a taper. When the navigation catheter 300 is inserted through the catheter 200, this distal end region 346 is configured to extend beyond and protrude out through the distal-most end 215 of the luminal portion 222 whereas the more proximal region of the body 360 (i.e. the first length described above) remains within the luminal portion 222.

As mentioned, the distal-most end 215 of the luminal portion 222 can be blunt and have no change in the dimension of the outer diameter whereas the distal end region 346 can be tapered providing an overall elongated tapered geometry of the catheter system. The outer diameter of the elongate body 360 also approaches the inner diameter of the luminal portion 222 such that the step-up from the elongate body 360 to the outer diameter of the luminal portion 222 is minimized. Minimizing this step-up prevents issues with the lip formed by the distal end of the luminal portion 222 catching on the tortuous neurovasculature, such as around the carotid siphon near the ophthalmic artery branch, when the distal end region 346 in combination with the distal end region of the catheter 200 bends and curves along within the vascular anatomy. In some implementations, the inner diameter of the luminal portion 222 can be at least about 0.052″ (1.321 mm), about 0.054″ (1.372 mm) and the maximum outer diameter of the elongate body 360 can be about 0.048″ (1.219 mm) such that the difference between them is about 0.006″ (0.1524 mm). In some implementations, the inner diameter of the luminal portion 222 can be about 0.070″ (1.778 mm) and the maximum outer diameter of the elongate body 360 can be about 0.062″ (1.575 mm) such that the difference between them is about 0.008″ (0.2032 mm). In some implementations, the inner diameter of the luminal portion 222 can be about 0.088″ (2.235 mm) and the maximum outer diameter of the elongate body 360 can be about 0.080″ (2.032 mm) such that the difference between them is about 0.008″ (0.2032 mm). In some implementations, the inner diameter of the luminal portion 222 can be about 0.072″ (1.829 mm) and the maximum outer diameter of the elongate body 360 is about 0.070″ (1.778 mm) such that the difference between them is only 2 thousandths of an inch (0.002″/0.0508 mm). In other implementations, the maximum outer diameter of the elongate body 360 is about 0.062″ (1.575 mm) such that the difference between them is about 0.010″ (0.254 mm). Despite the outer diameter of the elongate body 360 extending through the lumen of the luminal portion 222, the luminal portion 222 and the elongate body 360 extending through it in co-axial fashion are flexible enough to navigate the tortuous anatomy leading to the level of M1 or M2 arteries without kinking and without damaging the vessel.

The dimensions provided herein are approximate and each dimensions may have an engineering tolerance or a permissible limit of variation. Use of the term “about,” “approximately,” or “substantially” are intended to provide such permissible tolerance to the dimension being referred to. Where “about” or “approximately” or “substantially” is not used with a particular dimension herein that that dimension need not be exact.

The length of the tapered distal end region 346 can vary. In some implementations, the length of the distal end region 346 can be in a range of between about 0.50 cm to about 4.0 cm from the distal-most end of the elongate body 360 or between about 1.0 cm to about 3.0 cm. In other implementations, the length of the distal end region 346 is between 2.0 cm to about 2.5 cm. In some implementations, the length of the distal end region 346 varies depending on the inner diameter of the catheter 200 with which the navigation catheter 300 is to be used. For example, the length of the distal end region 346 can be as shorter (e.g., 1.2 cm) for a navigation catheter 300 sized to be used with a catheter 200 having an inner diameter of about (1.372 mm) and can be longer (e.g., 2.5 cm) for a navigation catheter 300 sized to be used with a catheter 200 having an inner diameter of about 0.088″ (2.235 mm). The distal end region 346 can be a constant taper from the larger outer diameter of the elongate body 360 down to a second smaller outer diameter at the distal-most terminus. In some implementations, the constant taper of the distal end region 346 can be from about 0.048″ outer diameter down to about 0.031″ (0.787 mm) outer diameter over a length of about 1 cm. In some implementations, the constant taper of the distal end region 346 can be from 0.062″ (1.575 mm) outer diameter to about 0.031″ (0.787 mm) outer diameter over a length of about 2 cm. In still further implementations, the constant taper of the distal end region 346 can be from 0.080″ (2.032 mm) outer diameter to about 0.031″ (0.787 mm) outer diameter over a length of about 2.5 cm. The length of the constant taper of the distal end region 346 can vary, for example, between 0.8 cm to about 2.5 cm, or between 1 cm and 3 cm, or between 2.0 cm and 2.5 cm. The angle of the taper can vary depending on the outer diameter of the elongate body 360. For example, the angle of the taper can be between 0.9 to 1.6 degrees relative to horizontal. The angle of the taper can be between 2-3 degrees from a center line of the elongate body 360. The length of the taper of the distal end region 346 can be between about 5 mm to 20 mm or about 20 mm to about 50 mm.

The lumen 368 of the elongate body 360 of the navigation catheter 300 can have an inner diameter that does not change over the length of the elongate body even in the presence of the tapering of the distal end region 346. Thus, the inner diameter of the lumen 368 extending through the tubular portion of the navigation catheter 300 can remain uniform and the wall thickness of the distal end region 346 can decrease to provide the taper. The wall thickness can thin distally along the length of the taper. Thus, the material properties in combination with wall thickness, angle, length of the taper can all contribute to the overall maximum flexibility of the distal-most end of the distal end region 346. The navigation catheter 300 undergoes a transition in flexibility from the distal-most end towards the snug point where it achieves an outer diameter that is no more than about 0.010″ (0.254 mm) different from the inner diameter of the catheter 200.

The length of the taper can also vary depending on the anatomy of the target region. The distal end region 346 can achieve its soft, atraumatic and flexible characteristic due to a material property other than due to a change in outer dimension to facilitate endovascular navigation to an embolus in tortuous anatomy. Additionally or alternatively, the distal end region 346 of the elongate body 360 can have a transition in flexibility along its length. The most flexible region of the distal end region 346 can be its distal terminus. Moving along the length of the distal end region 346 from the distal terminus towards a region proximal to the distal terminus. For example, the distal end region 346 can be formed of a material having a Shore material hardness of no more than 35D or about 62A and transitions proximally to be less flexible near where it is formed of a material having a material hardness of no more than 55D and 72D up to the proximal extension 366, which can be a stainless steel hypotube, or a combination of a material property and tapered shape. The materials used to form the regions of the elongate body 360 can include PEBAX (such as PEBAX 25D, 35D, 55D, 69D, 72D) or a blend of PEBAX (such as a mix of 25D and 35D, 25D and 55D, 25D and 72D, 35D and 55D, 35D and 72D, 55D and 72D, where the blend ratios may range from 0.1% up to 50% for each PEBAX durometer), with a lubricious additive compound, such as silicone or Mobilize (Compounding Solutions, Lewiston, Maine). In some implementations, the material used to form a region of the elongate body 360 can be Tecothane 62A. Incorporation of a lubricious additive directly into the polymer elongate body means incorporation of a separate lubricious liner, such as a PTFE, FEP, or HDPE liner, is unnecessary. This allows for a more flexible element that can navigate the distal cerebral anatomy and is less likely to kink. Similar materials can be used for forming the distal luminal portion 222 of the catheter 200 providing similar advantages. The flexibility of the distal end region 346 can be achieved by a combination of flexible lubricious materials and tapered shapes. For example, the length of the distal end region 346 can be kept shorter than 2 cm-3 cm, but maintain optimum deliverability due to a change in flexible material from distal-most end 325 towards a more proximal region a distance away from the distal-most end 325. In an implementation, the elongate body 360 is formed of PEBAX (polyether block amide) embedded silicone designed to maintain the highest degree of flexibility. The wall thickness of the distal end of the luminal portion 222 can also be made thin enough such that the lip formed by the distal end of the luminal portion 222 relative to the elongate body 360 is minimized.

The elongate body 360 has a benefit over a microcatheter in that it can have a relatively large outer diameter that is just 0.003″-0.010″ (0.0762 mm-0.254 mm) smaller than the inner diameter of the distal luminal portion 222 of the catheter 200 and still maintaining a high degree of flexibility for navigating tortuous anatomy. When the gap between the two components is too tight (e.g., less than about 0.003″ (0.0762 mm), the force needed to slide the navigation catheter 300 relative to the catheter 200 can result in damage to one or both of the components and increases risk to the patient during the procedure. The gap results in too tight of a fit to provide optimum relative sliding. When the gap between the two components is too loose (e.g., greater than about 0.010″/0.254 mm), the distal end of the catheter 200 forms a lip that is prone to catch on branching vessels during advancement through tortuous neurovasculature, such as around the carotid siphon where the ophthalmic artery branches off and the piston effect of withdrawal of the elongate body 360 can be decreased or lost.

The gap in ID/OD between the elongate body 360 and the distal luminal portion 222 can be in this size range (e.g., 0.003″-0.015″ (0.0762 mm-0.381 mm) or between 0.006″-0.010″ (0.152 mm-0.254 mm)) along a majority of their lengths. For example, the elongate body 360 can have a relatively uniform outer diameter that is between about 0.048″ (1.219 mm) to about 0.080″ (2.032 mm) from a proximal end region to a distal end region up to a point where the taper of the distal end region 346 begins. Similarly, the distal luminal portion 222 of the catheter 200 can have a relatively uniform inner diameter that is between about 0.054″ (1.372 mm) to about 0.088″ (2.235 mm) from a proximal end region to a distal end region. As such, the difference between their respective inner and outer diameters along a majority of their lengths can be within this gap size range of 0.003″ to 0.015″ (0.0762 mm-0.381 mm). The distal end region 346 of the elongate body 360 that is tapered will have a larger gap size relative to the inner diameter of the distal luminal portion 222. During use, however, this tapered distal end region 346 is configured to extend distal to the distal end of the catheter 200 such that the region of the elongate body 360 having an outer diameter sized to match the inner diameter of the distal luminal portion 222 is positioned within the lumen of the catheter 200 such that it can minimize the lip at the distal end of the catheter 200.

The elongate body 360 can be formed of various materials that provide a suitable flexibility and lubricity. Example materials include high density polyethylene, 77A PEBAX, 33D PEBAX, 42D PEBAX, 46D PEBAX, 54D PEBAX, 69D PEBAX, 72D PEBAX, 90D PEBAX, and mixtures thereof or equivalent stiffness and lubricity material. In some implementations, the elongate body 360 is an unreinforced, non-torqueing catheter having a relatively large outer diameter designed to fill the lumen it is inserted through and a relatively small inner diameter to minimize any gaps at a distal-facing end of the device. In other implementations, at least a portion of the elongate body 360 can be reinforced to improve navigation and torqueing (e.g. braided reinforcement layer). The flexibility of the elongate body 360 can increase towards the distal end region 346 such that the distal region of the elongate body 360 is softer, more flexible, and articulates and bends more easily than a more proximal region. For example, a more proximal region of the elongate body can have a bending stiffness that is flexible enough to navigate tortuous anatomy, such as the carotid siphon, without kinking. If the elongate body 360 has a braid reinforcement layer along at least a portion of its length, the braid reinforcement layer can terminate a distance proximal to the distal end region 346. For example, the distance from the end of the braid to the distal-most end 325 can be about 10 cm to about 15 cm or from about 4 cm to about 10 cm or from about 4 cm up to about 15 cm.

As mentioned, the elongate body 360 can be generally tubular along at least a portion of its length such that the single lumen 368 extends parallel to a longitudinal axis of the navigation catheter 300 (see FIGS. 3A-3C). In an implementation, the single lumen 368 of the elongate body 360 is sized to accommodate a guidewire 500, however use of the navigation catheter 300 generally eliminates the need for a guidewire lead. Preferably, the assembled system includes no guidewire. Guidewires are designed to be exceptionally flexible so that they deflect to navigate the severe turns of the anatomy. However, many workhorse guidewires have a stiffness along their longitudinal axis and/or are small enough in outer diameter that they find their own paths through an embolus rather than slipping around the embolus. In some cases, these guidewires can cause perforations and/or dissections of the vessel itself. Thus, even though the guidewire may have an outer diameter at its distal tip region that is small and very flexible at the distal tip, guidewires typically are incapable of atraumatically probing an embolus. Guidewires do not deflect upon encountering the dense proximal face of the embolus. Instead, guidewires embed and penetrate an embolus. The navigation catheter 300 has a softness, taper, and sizing that finds and/or creates space to slide between a portion of the embolus and the vessel wall rather than penetrating through it like a guidewire does. Methods of using the navigation catheter 300 without a guidewire or with a guidewire 500 parked within the lumen 368 acting as a rescue guidewire to deliver a catheter to distal regions of the brain.

The guidewire 500 can extend through the single lumen 368 generally concentrically from a proximal opening to a distal opening 326 at the distal-most end 325 of the navigation catheter 300. In some implementations, the proximal opening is at the proximal end of the navigation catheter 300 such that the navigation catheter 300 is configured for over-the-wire (OTW) methodologies. In other implementations, the proximal opening is a rapid exchange opening through a wall of the navigation catheter 300 such that the navigation catheter 300 is configured for rapid exchange rather than or in addition to OTW. In this implementation, the proximal opening extends through the sidewall of the elongate body 360 and is located a distance away from a proximal tab or luer 364 and distal to the proximal extension 366. The proximal opening can be located a distance of about 10 cm from the distal end region 346 up to about 20 cm from the distal end region 346. In some implementations, the proximal opening can be located near a region where the elongate body 360 is joined to the proximal extension 366, for example, just distal to an end of the hypotube. In other implementations, the proximal opening is located more distally, such as about 10 cm to about 18 cm from the distal-most end of the elongate body 360. A proximal opening that is located closer to the distal end region 346 allows for easier removal of the navigation catheter 300 from the catheter 200 leaving the guidewire in place for a “rapid exchange” type of procedure. Rapid exchanges can rely on only a single person to perform the exchange. The navigation catheter 300 can be readily substituted for another device using the same guidewire that remains in position. The single lumen 368 of the elongate body 360 can be configured to receive a guidewire in the range of 0.014″ (0.356 mm) and 0.018″ (0.457 mm) diameter, or in the range of between 0.014″ and 0.022″ (0.356 mm-0.559 mm). In this implementation, the inner luminal diameter of the elongate body 360 can be between 0.020″ and 0.024″ (0.508 mm-0.610 mm). The guidewire, the navigation catheter 300, and the catheter 200 can all be assembled co-axially for insertion through the working lumen of the guide sheath 400. The inner diameter of the lumen 368 of the elongate body 360 can be 0.019″ to about 0.021″ (0.483 mm-0.533 mm). The distal opening from the lumen 368 can have an inner diameter that is between about 0.018″ to about 0.024″ (0.457 mm-0.610 mm).

The region near the distal end region 346 can be tapered such that the outer diameter tapers over a length of about 1 cm to about 4 cm. In some implementations, the distal taper length is about 2.5 cm. In other implementations, the distal taper is over a length of about cm to about 5 cm, or about 1 cm to 4 cm, or about 2 cm to about 3 cm. The larger outer diameter can be at least about 1.5 times, 2 times, 2.5 times, or about 3 times larger than the smaller outer diameter. The distal end region 346 can taper along a distance from a first outer diameter to a second outer diameter, the first outer diameter being at least 1.5 times the second outer diameter. In some implementations, the distal end region 346 tapers from about 0.080″ (2.032 mm) to about 0.031″ (0.787 mm). In some implementations, the smaller outer diameter at a distal end of the taper can be about 0.026″ (0.66 mm) up to about 0.040″ (1.016 mm) and the larger outer diameter proximal to the taper is about 0.062″ (1.575 mm) up to about 0.080″ (2.032 mm). Also, the distal end region 346 can be formed of a material having a material hardness (e.g. 62A and 35D) that transitions proximally towards increasingly harder materials having (e.g., 55D and 72D) up to the proximal extension 366. A first segment of the elongate body 360 including the distal end region 346 can be formed of a material having a material hardness of and a length of about 10 cm to about 12.5 cm. The first segment of the elongate body 360 including the distal end region 346 can be formed of a material having a material hardness of 62A and a length of about 10 cm to about 12.5 cm. A second segment of the elongate body 360 can be formed of a material having a material hardness of 55D and have a length of about 5 cm to about 8 cm. A third segment of the elongate body 360 can be formed of a material having a material hardness of 72D can be about 25 cm to about 35 cm in length. The three segments combined can form an insert length of the elongate body 360 from where the proximal extension 366 couples to the elongate body 360 to the terminus of the distal end region 346 that can be about 49 cm in length.

The navigation catheter 300 has a flexible, distal embolus-probing tip section 346 that has a length in the range of 1 cm to 5 cm that tapers from a proximal outer diameter (e.g., about 1.58 mm-about 2.03 mm) to a distal outer diameter (e.g., about 0.66 mm-about 0.79 mm). The atraumatic tip region 346 is preferably radiopaque. The tapered tip region 346 has a flexibility allowing it to deflect generally away from a dense embolus towards the vessel wall. The deflection occurs upon advancement of the navigation catheter through the vessel on encountering a resistance to further axial motion from a generally organized or dense embolus within a flexible vessel having an inner diameter about 2-5 mm for an embolus located in the MCA or larger inner diameter up to about 8 mm for an embolus located proximal to the MCA, such as within the ICA. The tip region 346 is arranged to deflect away from the proximal face of the embolus towards the vessel wall and, in some instances, to move at least partially under the proximal face of the embolus so that between about 0 mm to about 3 cm of the embolus-probing tip section extends between the obstacle and the vessel wall upon application of an additional force to urge the embolus-probing tip section against the embolus. Conventional catheters and guidewires have a tip structure that tend to embed into the embolus as opposed to probe the front face of the embolus to find a space or deflect away from the proximal face. Guidewires have small outer diameters and flexible distal tips. Despite the small outer diameter and the flexibility, a guidewire tip is incapable of probing the embolus according to the methods provided herein. Rather, a guidewire tip construction, particularly when used with a microcatheter that provides a centering effect on the guidewire, results in the guidewire penetrating and embedding into or passing through the embolus, which is preferably avoided in aspiration thrombectomy. The tapered distal tip region 346 of the navigation catheter 300 probes the embolus so that the tip deflects and slips between the proximal face of the embolus and the vessel wall.

The distal end region of the guidewire has a profile that is much smaller compared to the profile of the distal tip region 346 of the navigation catheter. The outer diameter of the guidewire also stays small moving proximally along its length compared to the navigation catheter that enlarges to an even larger outer diameter moving proximally just a few centimeters. In turn, the force per unit area for the guidewire is much higher compared to the navigation catheter. A guidewire used in the neurovasculature, particularly at the level of the MCA, may have an outer diameter at the distal end that is 0.014″ (0.36 mm) and have a distal-facing contact area that is about 1.50×10⁻⁴ square inch (0.100 mm²). The outer diameter of the distal end of the navigation catheter can be about 0.031″ (0.79 mm) and the inner diameter of the distal end of the navigation catheter can be about 0.021″ (0.53 mm). The distal-facing contact area for the navigation catheter can be about 8.00×10⁻⁴ square inch (0.5 mm²) if the lumen is filled with a column of fluid and/or a guidewire. The distal-facing contact area for the navigation catheter can be about 4.20×10⁻⁴ square inch (0.27 mm²) for just the annular distal-facing surface without a column of fluid or guidewire within the lumen. Regardless, the force per unit area of the guidewire is significantly greater (i.e., about 2 to 5 times greater) than the force per unit area of the navigation catheter. The force per unit area of a 0.014″ guidewire for 1 N force is about 6,700 N/square inch (10 N/mm²) whereas the force per unit area of the navigation catheter is about 1,300 N/square inch (2 N/mm²) to about 2,400 N/square inch (4 N/mm²). The profile of the guidewire, in combination with the force per unit area for the guidewire (and centering effect provided by the microcatheter), creates a higher risk of penetration of the embolus rather than deflection upon encountering the proximal face of the embolus. The profile of the navigation catheter including the greater outer diameter as the distal end, the relatively short taper to an even larger outer diameter, and its high flexibility results in the navigation catheter being incapable of penetrating the embolus and instead deflecting away from the proximal face of the embolus upon encountering one within a vessel. Guidewires penetrate an embolus or vessel wall. The navigation catheter, in contrast, probes and deflects away from the embolus, finds any space and wedges into a final resting spot without penetrating the embolus or the vessel wall.

It is desirable to have a specially constructed tip region to ensure the tip region will deflect relative to an embolus, not penetrate the embolus or a vessel wall, when encountering it. The tip region will deflect until it finds a path or space. This is achieved by having a sufficient degree of flexibility of the fully polymeric distal tip region that includes a taper over a length so that the tip region deflects readily upon coming into contact with the proximal face of an embolus. The flexibility and shape of the tapered tip region results in the tip region, which is protruding from the aspiration catheter during advancement through the vessel, passing through less organized or less dense thrombotic material until the tip region encounters the true proximal face of the embolus. The tip region then deflects away from the organized or dense portion of the embolus so that, for example, it wedges between the embolus and the vessel wall. The tip region is constructed to find the path of least resistance in an atraumatic manner without being so flexible or prone to bending that it folds over onto itself and cannot be advanced.

The distal-most tip of the tip region can have a smooth, relatively rounded shape having a low friction outer surface that tends to encourage deflection of the tip region relative to the proximal face of the embolus. The distal tip can also be radiopaque due to embedding a material within the polymer as described in more detail below.

The navigation catheter 300 can incorporate a reinforcement layer. The reinforcement layer can be a coil, braid or other type of reinforcement to bridge the components of the navigation catheter 300 having such differences in flexibility. A braided reinforcement layer can bridge the transition from the rigid, proximal extension 366 to the flexible elongate body 360. In some implementations, the reinforcement layer can be a braid positioned between inner and outer layers of PEBAX. The reinforcement layer can terminate a distance proximal to the distal end region 346. The distal end region 346 can be formed of a material having a material hardness of at most about 35D. The first segment can be unreinforced polymer having a length of about 4 cm up to about 12.5 cm without metal reinforcement. The third segment of the elongate body 360 located proximal to the first segment can include the reinforcement layer and can extend a total of about 37 cm up to the unreinforced distal segment. A proximal end region of the reinforcement layer can overlap with a distal end region of the proximal extension 366 such that a small overlap of hypotube and reinforcement exists near the transition between the proximal extension 366 and the elongate body 360.

An entry port for a procedural guidewire can be positioned a distance away from the distal-most end of the elongate body 360. In some implementations, the entry/exit port can be about 18 cm from the distal-most end creating a rapid exchange wire entry/exit segment. The outer diameter of the elongate body 360 within the first two segments can be about 0.080″-0.082″ (2.032 mm-2.083 mm) whereas the third segment proximal to this rapid exchange wire entry/exit segment can have a step-down in outer diameter, such as about 0.062″-0.064″ (1.575 mm-1.626 mm).

In other implementations, the entire navigation catheter 300 can be a tubular element configured to receive a guidewire through both the proximal extension 366 as well as the elongate body 360. For example, the proximal extension 366 can be a hypotube or tubular element having a lumen that communicates with the lumen 368 extending through the elongate body 360 (shown in FIG. 1C). In some implementations, the proximal extension 366 can be a skived hypotube of stainless steel coated with PTFE having an outer diameter of 0.026″ (0.660 mm). In other implementations, the outer diameter can be between 0.024″ (0.610 mm) and (0.762 mm). In some implementations, such as an over-the-wire version, the proximal extension 366 can be a skived hypotube coupled to a proximal hub or luer 364. The proximal extension 366 can extend eccentric or concentric to the distal luminal portion 222. The proximal extension 366 can be a stainless steel hypotube. The proximal extension 366 can be a solid metal wire that is round or oval cross-sectional shape. The proximal extension 366 can be a flattened ribbon of wire having a rectangular cross-sectional shape. The ribbon of wire can be curved into a circular, oval, c-shape, or quarter circle, or other cross-sectional shape along an arc. The proximal extension 366 can have any of variety of cross-sectional shapes whether or not a lumen extends therethrough, including a circular, oval, C-shaped, D-shape, or other shape. In some implementations, the proximal extension 366 is a hypotube having a D-shape such that an inner-facing side is flat and an outer-facing side is rounded. The rounded side of the proximal extension 366 can be shaped to engage with a correspondingly rounded inner surface of the sheath 400. The hypotube can have a lubricious coating, such as PTFE. The hypotube can have an inner diameter of about 0.021″ (0.533 mm), an outer diameter of about 0.0275″ (0.699 mm), and an overall length of about 94 cm providing a working length for the navigation catheter 300 that is about 143 cm. Including the proximal luer 364, the navigation catheter 300 can have an overall length of about 149 cm. In some implementations, the hypotube can be a tapered part with a length of about 100 mm, starting proximal with a thickness of 0.3 mm and ending with a thickness of 0.10 mm to 0.15 mm. In still further implementations, the elongate body 360 can be a solid element coupled to the proximal extension 366 having no guidewire lumen.

The proximal extension 366 is shown in FIG. 3A as having a smaller outer diameter compared to the outer diameter of the elongate body 360. The proximal extension 366 need not step down in outer diameter and can also have the same outer diameter as the outer diameter as the elongate body 360. For example, the proximal extension 366 can incorporate a hypotube or other stiffening element that is coated by one or more layers of polymer resulting in a proximal extension 366 having substantially the same outer diameter as the elongate body 360.

At least a portion of the solid elongate body 360, such as the elongate distal end region 346, can be formed of or embedded with or attached to a malleable material that skives down to a smaller dimension at a distal end. The distal end region 346 can be shaped to a desired angle or shape similar to how a guidewire may be used. The malleable length of the elongate body 360 can be at least about 1 cm, 3 cm, 5 cm, and up to about 10 cm, 15 cm, or longer. In some implementations, the malleable length can be about 1%, 2%, 5%, 10%, 20%, 25%, 50% or more of the total length of the elongate body 360. In some implementations, the navigation catheter 300 can have a working length of about 140 cm to about 143 cm and the elongate body 360 can have an insert length of about 49 cm. The insert length can be the PEBAX portion of the elongate body 360 that is about 49.5 cm. As such, the malleable length of the elongate body 360 can be between about 0.5 cm to about 25 cm or more. The shape change can be a function of an operator manually shaping the malleable length prior to insertion in the patient or the distal end region 346 can be pre-shaped at the time of manufacturing into a particular angle or curve. Alternatively, the shape change can be a reversible and actuatable shape change such that the distal end region 346 forms the shape upon activation by an operator such that the distal end region 346 can be used in a straight format until a shape change is desired by the operator. The navigation catheter 300 can also include a forming mandrel extending through the lumen of the elongate body 360 such that a physician at the time of use can mold the distal end region 346 into a desired shape. As such, the moldable distal end region 346 can be incorporated onto an elongate body 360 that has a guidewire lumen.

The elongate body 360 can extend along the entire length of the catheter 200, including the distal luminal portion 222 and the proximal extension 230 or the elongate body 360 can incorporate the proximal extension 366 that aligns generally side-by-side with the proximal extension 230 of the catheter 200. The proximal extension 366 of the elongate body 360 can be positioned co-axial with or eccentric to the elongate body 360. The proximal extension 366 of the elongate body 360 can have a lumen extending through it. Alternatively, the portion 366 can be a solid rod or ribbon having no lumen.

Again with respect to FIGS. 3A-3C, like the distal luminal portion 222 of the catheter 200, the elongate body 360 can have one or more radiopaque markers 344 along its length. The one or more markers 344 can vary in size, shape, and location. One or more markers 344 can be incorporated along one or more parts of the navigation catheter 300, such as a tip-to-tip marker, a tip-to-taper marker, an RHV proximity marker, a Fluoro-saver marker, or other markers providing various information regarding the relative position of the navigation catheter 300 and its components. The tubular portion 360 of the navigation catheter 300 can have a radiopaque marker band embedded within or positioned over a wall of the tubular portion 360 near the distal end region 346. In some implementations and as best shown in FIG. 3C, a distal end region can have a first radiopaque marker 344a and a second radiopaque marker 344b can be located to indicate the border between the tapering of the distal end region 346 and the more proximal region of the elongate body 360 having a uniform or maximum outer diameter. A first radiopaque marker band 344a can be found at the distal end of the tapered distal end region 346 and a second radiopaque marker band 344b can be found at the proximal end of the tapered distal end region 346. The proximal radiopaque marker band 344b can have a proximal edge, a distal edge, and a width between the proximal and distal edges. This provides an operator and/or the robotic drive system 600 with information regarding an optimal extension of the distal end region 346 relative to the distal end of the luminal portion 222 to minimize the lip at this distal end of the luminal portion 222 for advancement through tortuous anatomy. When in the advancement configuration, the proximal edge of the radiopaque marker band 344b can align substantially with the distal end of the distal, catheter portion 222 such that the radiopaque marker band 344b remains external to the lumen 223 of the distal, catheter portion 222. At least a portion of the radiopaque marker band 344b can be positioned at the snug point, or the point of the navigation catheter 300 where the outer diameter is no more than about 0.010″ (0.254 mm), preferably between about 0.006″ and 0.008″ (0.152 mm-0.203 mm) smaller than the inner diameter of the catheter 200 it is positioned within. The at least one snug point of the tubular portion 360 can be located proximal to the distal end region 346 and can be where the taper of the distal end region 346 substantially ends. This allows for full extension of the tapered distal end region 346 outside the distal end of the catheter 200 and the snug point aligned substantially within the distal opening 231 from the lumen 223 of the distal, catheter portion 222 thereby minimizing any distal-facing lip that might be created by the catheter 200. The snug point can be located along at least a portion of a length of the outer diameter of the tubular portion 360 that has a length of at least about 5 cm up to about 10 cm, the outer diameter being substantially uniform or non-uniform.

The distal end region 346 can be a constant taper from the larger outer diameter of the elongate body 360 (e.g., the distal end of the marker 344b) down to a second smaller outer diameter at the distal-most terminus (e.g., the proximal end of the marker 344a). In other implementations, for example where the distal end region 346 is not necessarily tapered, but instead has a change in overall flexibility along its length, the second radiopaque marker 344b can be located to indicate the region where the relative flexibilities of the elongate body 360 (or the distal end region 346 of the elongate body 360) and the distal end of the luminal portion 222 are substantially the same. The marker material may be a platinum/iridium band, a tungsten, platinum, or tantalum-impregnated polymer, or other radiopaque marker that does not impact the flexibility of the distal end region 346 and elongate body 360. In some implementations, the radiopaque markers are extruded PEBAX loaded with tungsten for radiopacity.

The distal marker 344a near the distal-most end 325 of the navigation catheter 300 can be differentiated from the distal marker 224a on the catheter 200 by its characteristic appearance under fluoroscopy as well as by simply jogging back and forth the atraumatic navigation catheter 300 to understand the relationship and positioning of the navigation catheter 300 relative to the catheter 200. The second marker 344b on the navigation catheter 300 that is proximal to the distal-most tip marker 344a can delineate the taper of the distal end region 346, i.e. where the outer diameter of the navigation catheter 300 has a sufficient size to reduce the “lip” of the transition between the navigation catheter 300 and the catheter 200 through which it is inserted and configured to deliver. The markers aid in positioning the navigation catheter 300 relative to the distal end 215 of the aspiration catheter 200 such that the tip 215 of the catheter 200 is aligned with the taper of the navigation catheter 300 and the best alignment is facilitated. In some implementations, the proximal marker band can be about 2.0 mm wide and the distal marker band can be about 2.5 mm wide to provide discernable information about the distal end region 346.

The relationship between the distal tip marker 224 of the aspiration catheter 200 is at or ideally just proximal to the taper marker 344b of the navigation catheter 300 (i.e., the proximal marker or proximal end of distal taper marker, if just one marker incorporated to identify the start of the taper) is identifiable with the tandem marker system. The paired elements 224, 344b are in a “tip-to-taper” position. The relative extension between the navigation catheter 300 and the catheter 200 can be adjusted at the insertion of the system into the RHV. However, the relative extension can become altered with advancement through the sheath or guide catheter. As the system exits the guide catheter, the aspiration catheter 200 and the navigation catheter 300 can be adjusted to that the tip-to-taper position is assumed as the system traverses the often tortuous proximal vessel (e.g., the cervical internal carotid artery) towards more distal targets. The system of the aspiration catheter 200 and the navigation catheter 300 can be locked into their relative extension so that the juxtaposition of the navigation catheter 300 and the aspiration catheter 200 is maintained. As the aspiration catheter 200 is visualized within the sheath distal end or even slightly beyond the distal end of the sheath, the navigation catheter 300 can be adjusted to assume the proper position relative to the catheter before advancement resumes. The optimum relative extension between the distal marker 224 of the catheter 200 to the taper marker 344b on the navigation catheter 300 can be maintained through as much of the anatomy as possible to maximize the delivery capability of the navigation catheter 300 to navigate both tortuosity and to avoid side branches, such as the ophthalmic artery. Once a desired site is reached, the navigation catheter 300 can be held fixed and the aspiration catheter 200 advanced over the navigation catheter 300 towards the embolus, but without crossing the embolus with the catheter 200. Alternatively, the navigation catheter 300 can be withdrawn proximally and the catheter 200 allowed to ride momentum of stored forces distally towards the embolus as described elsewhere herein. The withdrawal step of the navigation catheter 300 can be performed manually by an operator either directly at the location of the patient or via the control station 700 via the robotic drive system 600, or automatically via the robotic drive system 600.

The catheter 200 and navigation catheter 300 (with or without a guidewire) can be advanced as a single unit through the both turns of the carotid siphon. Both turns can be traversed in a single smooth pass to a target in a cerebral vessel without the step-wise adjustment of their relative extensions and without relying on the conventional step-wise advancement technique with conventional microcatheters. The catheter 200 having the navigation catheter 300 extending through it allows an operator (via the robotic drive system 600) to advance them in unison in the same relative position from the first bend of the siphon through the second bend beyond the terminal cavernous carotid artery into the ACA and MCA. Importantly, the advancement of the two components can be performed in a single smooth movement through both bends.

The navigation catheter 300 can be in a juxtapositioned relative to the catheter 200 that provides an optimum relative extension between the two components for single smooth advancement. The navigation catheter 300 can be positioned through the lumen of the catheter 200 such that its distal end region 346 extends just beyond a distal-most end 215 of the catheter 200. The distal end region 346 of the navigation catheter 300 eliminates the stepped transition between the inner member and the outer catheter 200 thereby avoiding issues with catching on branching vessels within the region of the vasculature such that the catheter 200 may easily traverse the multiple angulated turns of the carotid siphon. The optimum relative extension, for example, can be the distal end region 346 of the elongate body 360 extending just distal to a distal-most end 215 of the catheter 200. A length of the distal end region 346 extending distal to the distal-most end 215 of the catheter 200 during advancement can be between 0.5 cm and about 4 cm. This juxtaposition can be a locked engagement with a mechanical element, by an operator holding the two components together, and/or by the two components being held fixed by the robotic drive system 600.

The mechanical element can include coupling feature 250, such as a clip, clamp, c-shaped element or other connector, configured to receive the proximal portions of the catheters 200, 300. The coupling feature 250 can be configured to snap together with the proximal extension 366 through an interference fit such that a first level of force is needed in order to insert the proximal extension 366 into the clip of the tab 234 and a second, greater level of force is needed to remove the proximal extension 366 from the clip of the tab 234. However, upon inserting the proximal extension 366 into the coupling feature 250 the navigation catheter 300 and the catheter 200 can still be slidably adjusted relative to one another along a longitudinal axis of the system. The amount of force needed to slidably adjust the relative position of the two components can be such that inadvertent adjustment is avoided and the relative position can be maintained during use, but can be adjusted upon conscious modification. The configuration of the coupling between the proximal extension 366 of the navigation catheter 300 and the proximal control element 230 of the catheter 200 can vary. Generally, however, the coupling is configured to be reversible and adjustable while still providing adequate holding power between the two elements in a manner that is relatively user-friendly (e.g. allows for one-handed use) and organizes the proximal ends of the components (e.g. prevents the proximal control element 230 and proximal extension 366 from becoming twisted and entangled with one another). The coupling feature 250 configured to prevent entanglement and aid in the organization of the proximal portions can be integrated with the tabs or can be a separate feature located along their proximal end region.

The components can be advanced together with a guidewire 500, over a guidewire 500 pre-positioned, or without any guidewire at all. In some implementations, the guidewire 500 can be pre-assembled with the navigation catheter 300 and catheter 200 such that the guidewire 500 extends through a lumen 368 of the navigation catheter 300, which is loaded through the lumen 223 of the catheter 200, all prior to insertion into the patient. The pre-assembled components can be simultaneously inserted into the sheath 400 and advanced together up through and past the turns of the carotid siphon. The guidewire 500 may be located within the lumen 368 of the navigation catheter 300 and parked proximal of the tapered distal end region 346 or proximal of the distal tip 325 for potential use in the event the navigation catheter 300 without a guidewire does not reach the target location. For example, a distal tip of the guidewire can be positioned about 5 cm to about 40 cm, or about 20 cm to about 30 cm proximal of the distal end region 346 of the navigation catheter 300. At this location the guidewire 500 does not interfere with the performance or function of the navigation catheter. The guidewire 500 can be positioned within the lumen 368 of the navigation catheter 300 such that the distal end of the guidewire 500 is within the navigation catheter 300 during the step of advancing the assembled system of devices together and is extendable from the navigation catheter 300 out the distal opening 326 when needed for navigation. In one example, a rescue guidewire is parked within the lumen of the navigation catheter with a distal end of the guidewire about 0 cm to about 40 cm proximal or about 5 cm to about 35 cm proximal or about 7 cm to about 30 cm of the distal end of the navigation catheter, preferably about 10 cm proximal of the distal end of the navigation catheter. The guidewire 500 at this parked position can provide additional support for the proximal portion of the system without affecting the flexibility and performance of the distal portion of the system.

The use of the navigation catheter 300 with the tapered distal end region 346 allows for delivery of large bore aspiration catheters, even full-length “over-the-wire” catheters or catheters such as those described herein having a proximal extension. The navigation catheter 300 is designed specifically such that the catheter 200 can be delivered without a need for a guidewire. This ability to deliver the catheter 200 without a guidewire (or with a guidewire located within the lumen 368 of the navigation catheter 300 and parked proximal of the tapered distal end region 346 and/or proximal of the distal opening 326 for potential use) and without crossing the embolus is based, in part, upon the smooth transitions between the outer diameter of the navigation catheter 300 and the catheter 200 as well as the smooth transition in flexibility between the two. When the navigation catheter 300 is bent into an arc of greater than 180 degrees, the softness and flexibility creates a smooth arc without severe bends or kinks in the geometry of the catheter. Thus, the navigation catheter 300 seeks the larger lumens and goes where the majority of blood flow goes as opposed to into the smaller branch arteries. The distal end region 346 of the navigation catheter 300 can facilitate a strong preference to seek out the larger vessels during advancement into the distal vessels. This propensity to stay within the main channel allows for the advancement of large bore catheters without the aid of a guidewire. The propensity to follow the main channels of blood flow aligns with acute ischemic stroke pathophysiology where major emboli tend to follow these same routes to a point where the embolus lodges and interrupts antegrade blood flow. As well, these major channels are often ideal for placement of access catheters as these conduit arteries allow for smaller catheters to pass into specific target arteries for therapeutic intervention.

Standard neurovascular intervention, and nearly all endovascular intervention, is predicated on the concept that a guidewire leads a catheter to a target location. The guidewires are typically pre-shaped and often find side-branches of off-target locations where the guidewire will bunch or prolapse causing time-consuming nuisances during interventions that often require repeated redirection of the guidewire by the operator to overcome. In addition, this propensity of a guidewire to enter side-branches can be dangerous. Guidewires are typically 0.014″ to 0.018″ (0.356 mm-0.457 mm) in the neuroanatomy and will find and often traumatize small branches that accommodate this size, which can lead to small bleeds or dissections and occlusion. In a sensitive area like the brain these events can be catastrophic. The tendency of a guidewire to bunch and prolapse can also cause a leading edge to the guidewire that can be advanced on its own or as part of a tri-axial system to create dissection planes and traumatize small vessels. Guidewires are also designed to cross the embolus, primarily for the purpose of securing the guidewire to provide support for delivery of a catheter over the guidewire. However, crossing the embolus with the guidewire can increase a risk of dislodging embolic debris, which travels distal to the occlusion site.

In contrast, the navigation catheter 300 described herein preferentially stays in the larger lumen of a conduit vessel. In the setting of stroke treatment, an embolus is driven to certain anatomies because of the blood flow that the arterial system draws in the cerebral anatomy. The navigation catheter 300 tends to traverse a path identical to the path an embolus will take, particularly an embolus driven from a location, such as cardiac or carotid etiology. The navigation catheter 300 delivers to the largest lumen within the anatomy even in light of the highly tortuous anatomy and curves being navigated. The navigation catheter 300 can preferentially take the larger lumen at a bifurcation while also following the current of the greatest blood flow thereby maintaining the general direction and angulations of the parent vessel. In viewing the standard anatomy found in the cerebral vasculature, the Circle of Willis is fed by two vertebral and two carotid conduit arteries. As these four arteries are the access points to the cerebral anatomy—the course of the navigation catheter 300 can be identified and has been validated in standard cerebral anatomy models.

In the anterior circulation where the conduit artery point of entry for cerebral endovascular procedures is the internal carotid artery (ICA), the navigation catheter can guide the large-bore catheter to the M1 segment of the middle cerebral artery (MCA) bypassing the anterior communicating artery (ACA) and anterior temporal branch (ATB). The very flexible nature of the navigation catheter 300 combined with the distal flexible nature of most cerebral catheters combine to allow delivery through severe tortuosity. Independent of the tortuous nature of the course of the arteries, the navigation catheter 300 tends to navigate the turns and deliver to the largest offspring from a parent artery, for example, ICA to M1 segment of the MCA. The M2 level branching of the M1 can be variable, but is often seen to have two major M2 branches (superior and inferior) and, depending on the anatomy, which can vary significantly between patients, may be seen to bifurcate “equally” or “unequally.” If the caliber of the M2 branching is of similar size and angulation, the navigation catheter 300 may take one of the two branches. If the target for catheter placement is not in a favorable angulation or size of artery, the navigation catheter 300 may be curved (e.g., via shaping of a malleable distal tip) and directed or a guidewire may be used.

In some anatomies where the M2 bifurcation is “even” in size, a back-and-forth motion may aid in selecting one branch then the other while still avoid the need or use of a guidewire or a curved distal tip of the navigation catheter. The back-and-forth motion can allow for the navigation catheter to be directed into either branch of the M2. The navigation catheter, even when initially straight, achieves some curvature that aids in directing it into a branch vessel. Thus, when an operator encounters an M2 bifurcation and there is a desire to cannulate either branch of an evenly divided bifurcation, selection of either branch is possible using the navigation catheter without a guidewire.

Thus, main channels such as the ICA, the middle cerebral artery and its tributaries in the anterior circulation will naturally be the pathway of preference for the described navigation catheter and subsequence large-bore catheter delivery (via access from the ICA). A similar phenomenon can occur in the posterior circulation, which is accessed via the vertebral arteries arising from the subclavian arteries on the right and the left. The navigation catheter will take the main channels in this circulation as well by traversing the vertebral arteries to the basilar artery and to the major tributaries of the basilar: the posterior cerebral artery and superior cerebellar arteries in the posterior circulation.

Navigation using the navigation catheter can provide maximal deliverability with minimal vascular trauma. Catheters can cause “razoring” effects in a curved vessel because the blunt end of a large bore catheter can tend to take the greater curve in rounding a vessel when pushed by the operator. This blunt end can gouge or “razor” the greater curve with its sharp edge increasing the risk for dissection along an anatomic plane within the multilayered mid- or large-sized artery or vein (see, e.g. Catheter Cardiovasc. Interv. 2014 February; 83(2):211-20). The navigation catheter can serve to minimize the edge of these catheters. Positioning the navigation catheter within the lumen of the large-bore catheter such that the taper marker of the navigation catheter is aligned optimally with the distal tip marker of the catheter minimizes the edge and thereby eliminates “razoring” as the large-bore catheter is advanced through turns of the vessel. This is particularly useful for the cerebral anatomy. Stroke treatments are typically needed in regions distal to the carotid siphon, particularly distal to the ophthalmic artery takeoff from the greater curve of the severe tortuosity of the final turn of the carotid siphon “S-turn”, the “anterior genu” of the carotid siphon typically seen as part of the terminal internal carotid artery (ICA). The specifics of the navigation catheter in proper alignment within the large bore catheter (the “tip-to-taper” position noted by the distal tip marker) relative to the taper marker of the navigation catheter maximize the likelihood that razoring and hang-up on the ophthalmic artery are avoided during manual advancement of the catheter system. The taper marker of the navigation catheter can be positioned at or past the take-off of the ophthalmic artery to minimize these deleterious effects and allows the large-bore catheter to pass the ophthalmic artery without incident. In a relatively straight segment, which is common after passing the siphon, the large-bore catheter can be advanced over the navigation catheter, which serves still as a guiding element to the target. The transition between the navigation catheter and the distal edge of the large-bore catheter is insignificant, especially compared to the step changes present with a typical microcatheter or guidewire, which do not prevent hang-ups on branches, such as the ophthalmic artery. The navigation catheter allows for maneuvering of the large-bore catheter clear to the face of the embolus without use of a microcatheter or guidewire and without crossing and/or fragmenting the embolus in any way.

Robotic Drive System

FIG. 4A is a schematic view of the robotic drive system 600 having the proximal ends of a catheter system 150 and a guide sheath 400 installed within the cassette 605. As mentioned above with regard to FIG. 2, the robotic drive system 600 can include a base console 601 and a cassette 605 capable of mating with the console 601. The robotic drive system 600 can include a controller 610 having one or more inputs 612, outputs 614, and sensors 625. The cassette 605 can include sets of rollers 615 or other sort of manipulation mechanism and a plurality of connectors 620. The guide sheath 400 can be engaged by at least a first connector 620 in the form of a rotation gear. The sheath retainer sleeve 622 (shown in FIG. 2) can be advanced over the guide sheath 400 and connected to the access sheath introducer (also not shown) at the patient. The sheath retainer sleeve 622 is slidably attached to the cassette 605 to provide support when the guide sheath 400 is advanced, retracted, or rotated due to motion of the cassette 605 and/or the arm 603, or advancement of components of the catheter system 150 through the guide sheath 400. The guide sheath 400 can slidably move within the sheath retainer. The guide sheath 400 can move within the sheath retainer sleeve 622 via a slit extending along the length of the sheath retainer. The sheath retainer sleeve 622 may include a collapsible section or include multiple tubes that slide within each other to telescopically shorten and lengthen as the guide sheath 400 is inserted and retracted. The telescoping or collapsible tube(s) provide support during a procedure to prevent buckling or bowing.

The plurality of rollers 615 in cassette 605 manipulate the various components of the distal access system 100 when the components are loaded and latched into the cassette 605. The rollers 615 are positioned to optimally manipulate each component. For example, rollers that manipulate outer components can be positioned toward a distal side of cassette 605, rollers that manipulate middle components can be positioned more proximally, and rollers that manipulate inner components can be positioned even more proximally on a proximal side of the cassette 605. Rollers 615 associated with the inner-most component are positioned on the proximal end of cassette 605. In some cases, more than one set of rollers 615 can manipulate one component. For example, a first set of rollers 615a can manipulate a first region of catheter 200 (e.g., body 222 of catheter 200 within rollers 615a shown in FIG. 4A) and a second set of rollers 615b can manipulate a second region of the same catheter 200 (e.g., smaller diameter proximal control element 230 of the catheter 200 within rollers 615b shown in FIG. 4A). The roller set that controls movement of a component can be dependent upon the degree of extension of that component. For example, the first set of rollers 615a can manipulate the catheter 200 when the body 222 of the catheter 200 is withdrawn proximal to and positioned outside the hub 434 of guide sheath 400. The second set of rollers 615b can manipulate the catheter 200 when the body 222 of the catheter 200 is advanced distally and positioned fully inside the hub 434 of the guide sheath 400. The second set of rollers 625b can control the advancement and withdrawal of the catheter 200 once the first set of rollers 615a are no longer in contact with the catheter.

In some cases, the rollers 615 can be aligned with the longitudinal axis A. In other cases, the rollers 615 can be offset and at an angle from longitudinal access A, preferably at an angle 45 degrees or less. Additionally, some rollers can be arranged at an angle and also out of plane with the majority of rollers on cassette 605. The number, positioning and angulation (i.e., on-axis, angled, out of plane) of rollers can be configured to optimize the forward and backward movement of each component over the entire movement range of each component. In an example and still with respect to FIG. 4A, the cassette 605 can include a first set of rollers 615a located near the connector 620 such that the first set of rollers 615a is located proximal to the guide sheath hub 434. This first set of rollers 615a can be aligned so that a component driven through the rollers 615a is aligned with longitudinal axis A. The cassette 605 can include a second set of rollers 615b located proximal to the first set of rollers 615a. The second set of rollers 615b can be off-set from and at an angle relative to the longitudinal axis A. The second set of rollers 615b can also be off-plane to rollers 615a so a component coupled to rollers 615b is not in contact with rollers 615a. Alternately, when rollers 615b are active, rollers 615a may spring apart or otherwise disengage, so as not to interfere with movement of an inner component. A third set of rollers 615c can be located proximal to the first set of rollers 615a. Unlike the second set of rollers 615b, however, the third set of rollers 615c can be aligned with both the first set of rollers 615a and the longitudinal axis A. FIG. 4A shows a fourth set of rollers 615d that is located proximal to the third set of rollers 615c and aligned with the longitudinal axis.

Still with respect to FIG. 4A, a first catheter 200 can be inserted through the working lumen of the guide sheath 400 so that a distal end 215 of the first catheter 200 extends past the distal opening 408 of the guide sheath 400. The first catheter 200 can be received through the first set of rollers 615a located proximal of the hub 434 of the guide sheath 400. The second set of rollers 615b located proximal to the first rollers 615a and offset relative to a longitudinal axis A can engage the proximal control element 230 of the first catheter 200. The first catheter 200 can have a navigation catheter 300 extending through its single lumen so that the distal end region 346 extends distal to the distal end 215 of the first catheter 200. The third set of rollers 615c can engage a proximal extension 366 of the navigation catheter 300. The fourth set of rollers 615d can engage a guidewire 500 extending through the navigation catheter 300. The rollers 615d may also include a means to rotate the guidewire as well as advance and retract the guidewire.

The rollers can include soft elastomer that can engage different diameters of the component, such as thermoplastic elastomers like santoprene, silicone, urethane foam, neoprene, etc.

The number and arrangement of the roller sets can vary depending on whether the catheter system to be used includes 1, 2, 3, 4 or more components and/or have multiple diameters. The roller sets need not engage each component of the catheter system. In other words, the number of components being advanced and the number of roller sets can differ. In the example shown in FIG. 4A, the catheter system includes a first catheter 200, a navigation catheter 300 extending through the first catheter 200, and a guidewire 500 extending through the navigation catheter 300. The distal section 222 of the first catheter 200 can be engaged by the first roller set 615a and the proximal section 230 of the first catheter 200 engaged by the second roller set 615b. The first roller set 615a can be aligned with the longitudinal axis A and the second roller set 615b can be arranged at an angle to the longitudinal axis A. The navigation catheter 300 can be engaged by the third roller set 6154c located proximal to the first and second roller sets 615a, 615b. The guidewire 500 can be engaged by the fourth roller set 615d located proximal to the third roller set 615c near a proximal end region of the cassette 605. In this example, the system includes three components each having at least one dedicated roller set. However, the catheter 200 and navigation catheter 300 need not include their own dedicated roller sets. For example, a single roller set can move both components. In one implementation, the catheter 200 and navigation catheter 300 can be clipped or otherwise coupled together so as to be advanced/retracted together by a single roller set. In another implementation, the roller sets can be physically coupled to advance the two components. Thus, multiple components coupled together can be moved by the robotic system 600 using a single roller set as a single component is moved.

The same rollers may be used for advancing more than one component of catheter assembly 150. With reference again to FIG. 4A, in an initial state of advancement the larger diameter distal luminal portion 222 of the catheter 200 can be engaged by the first set of rollers 615a and the proximal portion 230 of the catheter 200 can be engaged by the second roller set 615b. At a second stage of advancement once the proximal end of the larger diameter distal luminal portion 222 of the catheter 200 has been advanced inside of the RHV 434 of the guide sheath 400, advancement of the catheter 200 can then be driven via the proximal control element 230 of the catheter 200. Advancement of the catheter 200 can be driven by the second set of rollers 615b engaged with the proximal control element 230 and the robotic drive system 600 transfer drive of the catheter 200 to that set of rollers 615b rather than the first set of rollers 615a. The first set of rollers 615a can be engage with and control movement of the inner navigation catheter 300 while the second, off-plane set of rollers 615b, still engaged with the proximal element 230, continues to control the movement of catheter 200. Alternatively, the third set of rollers 615c engaged with the inner navigation catheter 300 can continue to control and drive its movement. Any of a variety of combinations are considered herein.

Movement of the catheter 200 need not involve multiple rollers of the robotic drive system 600. Rather, the robotic drive system 600 can be configured to manipulate the catheter 200 with a single roller set engaged with the catheter 200, for example, only the proximal element 230. The catheter 200 can be first inserted into guide sheath 400 and advanced manually until the entire luminal portion 222 has entered the RHV 434 of the sheath 400. The proximal element 230 can then be inserted between rollers 615b to allow the robotic drive system 600 to control movements of the catheter 200. In this implementation, the robotic drive system 600 can include only one set of rollers 615b for the catheter 200 instead of two sets 615a, 615b as shown in FIG. 4A. FIG. 4A shows the roller set 615b located at an angle relative to the longitudinal axis A, however, the dedicated roller set for the catheter can be aligned with the longitudinal axis A as there is no need for rollers to be off-plane from the rest of the cassette rollers.

In an implementation, the catheter system includes two catheters 200 having sizes configured to allow one catheter to nest within the other catheter. For example, a first catheter can be a larger 0.088″ ID and a second catheter can be 0.070″ ID that has an OD sized to insert within the ID of the first catheter. The first and second catheters can be driven at the same time when the respective larger diameter portions (distal luminal portions 222) of the first and second catheters are extending outside of the guide sheath 400. The first catheter having the larger ID can be initially driven up to the targeted region in the vessel with the 0.070″ second catheter preloaded within it and moving together with the first catheter that is actively driven by a set of rollers. Then, if the smaller second catheter is needed for the procedure, the larger first catheter can be removed from the rollers and the proximal control element 230 of the second catheter inserted into the rollers to be drive directly. Once the first and second catheters are both inserted past their respective material transitions between the proximal control element 230 and the distal luminal portion 222, the second set of rollers can be used to control each of the first and second catheters independently.

FIG. 4B shows a robotic drive system 600 configured to manipulate nested distal access catheters 200a, 200b. The first catheter 200a can include a distal luminal portion 222a and a proximal control element 230a. The second catheter 200b extending through the first catheter 200a also can include a distal luminal portion 222b and a proximal control element 230b. A navigation catheter 300 can extend through the second catheter 200b and a guidewire 500 can extend through the navigation catheter 300. A first set of rollers 615a can drive the proximal control element 230a of the first catheter 200a. A second set of rollers 615b can drive the proximal control element 230b of the second catheter 200b nested within the first catheter 200a. The second catheter 200b can be physically clipped with the navigation catheter 300 such that the two are moved as a single unit by one roller set as described elsewhere herein. Alternatively, the system 600 can include a third set of rollers 615c that drives a proximal extension 366 of the navigation catheter 300. A still further set of rollers 615d can drive the guidewire 500. Depending on whether the catheter and the navigation catheter 300 are clipped together and moved as a unit, the system includes more components than roller sets. Where the navigation catheter 300 and guidewire 500 are shown extending along the longitudinal axis A and engaging their respective sets of rollers, one or both of the guidewire 500 and the navigation catheter 300 may also be off-set from the axis A and engage with a different set of rollers so that they come out at an angle from the cassette 605 as opposed to directly behind or through a proximal end of the cassette 605.

The catheters 200a, 200b can be initially advanced until their luminal portions 222a, 222b have entered the RHV 434 and before their proximal elements 230a, 230b are inserted into their respective rollers 615a, 615b. The initial advancement can be performed manually by a user or by a set of rollers. For example, a further set of rollers may be included that are located just proximal to the RHV 434 of the guide sheath 400, similar to what is shown in FIG. 4A. The additional set of rollers can be used to drive nested components, such as catheter 200a nested with catheter 200b. Once the proximal end of the larger diameter distal luminal portion 222a has been advanced inside of the RHV 434 of the guide sheath 400 (whether manually or via a further set of rollers), advancement of the catheter 200a can then be driven via engaging the proximal control element 230a of the catheter 200a with a roller set 615a. The smaller diameter proximal control element 230b can be manually transferred into the second set of rollers 615b and the robotic drive system 600 transfer drive of the catheter 200b to that set of rollers 615b rather than the first set of rollers. Alternatively, the outer catheter 200a can incorporate a slit that allows the distal luminal portion 222a to ride off to a side of the inner navigation catheter 300. This arrangement eliminates a need for a set of rollers directly proximal to the hub 434 of the guide sheath 400.

The rollers for the navigation catheter 300 can be physically linked to the rollers for the proximal control element of the catheter within which the navigation catheter 300 resides. For example, FIGS. 4A and 4C shows rollers 615b engaged with proximal control element 230 of catheter 200 physically linked to rollers 615c engaged with the navigation catheter 300. FIG. 4B shows rollers 615b engaged with proximal control element 230b of second catheter 200b physically linked to rollers 615c engaged with navigation catheter 300. The physical linkage allows for the catheter 200 to move simultaneously and synchronously with its navigation catheter 300 as the rollers are driven together. The linkage can be via a clip or other mechanical feature that joins the rollers. The drive system 600 rollers need not be synchronized to simultaneously advance two components. As described elsewhere herein, the catheter 200 can be mechanically clipped to the navigation catheter 300 as described above so that a single roller set can be used to advance them simultaneously.

Although the catheter systems described herein can be advanced without the use of a guidewire, the robotic drive system can include a further set of rollers 615d (see FIGS. 4A-4C) for engaging the guidewire 500. The further set of rollers 615d can be positioned even further proximally along the cassette 605 so as to engage with a guidewire 500, if used. The location of the guide wire rollers 615d allow for a full range of motion for the navigation catheter 300. The further set of rollers 615 can also be used to engage with an interventional device, such as a stent retriever, stent, aneurysm coil, flow diverter deliver system and the like.

The configuration of the rollers 615 can vary. Each roller 615 can include a friction element or tire mounted on a central shaft or axle. The rollers are configured to grip the components of the catheter system 150 without damaging the component such that the element moves when the rollers 615 rotate. The rollers can rotate in two directions so as to both advance and retract the element. At least one roller of a set of rollers can have a shaft that is driven to turn by a motor within the cassette 605 or console 601. This allows for the set of rollers to be used to drive a catheter body engaged between the set of rollers to move relative to the cassette 605. One roller of the set of rollers can be actively driven by the motor turning the shaft whereas the other roller of the set of rollers can passively turn on its shaft without being actively driven. Each set of rollers 615 can have at least one tire that is driven by a motor and a passive tire that turns without being driven by the motor.

The tires can be made of an elastomeric material that is somewhat tacky to aid in the engagement with a catheter body in order to urge the catheter body in a particular direction. The rollers can include soft elastomer, such as thermoplastic elastomers like santoprene, silicone, urethane foam, neoprene, etc. The material and configuration of the tires can be atraumatic so as to avoid damage to the catheter body when the catheter body is sandwiched between a set of rollers. In some implementations, the material of the tire can be a compressible elastomer so that the set of rollers are able to accommodate different outer diameters between them. In some implementations, one or more of the rollers 615 can include a compressible sleeve that allows the passage of one or more of the catheters when in the open state or secures the one or more catheters (or the guide wire 500) when in the closed state.

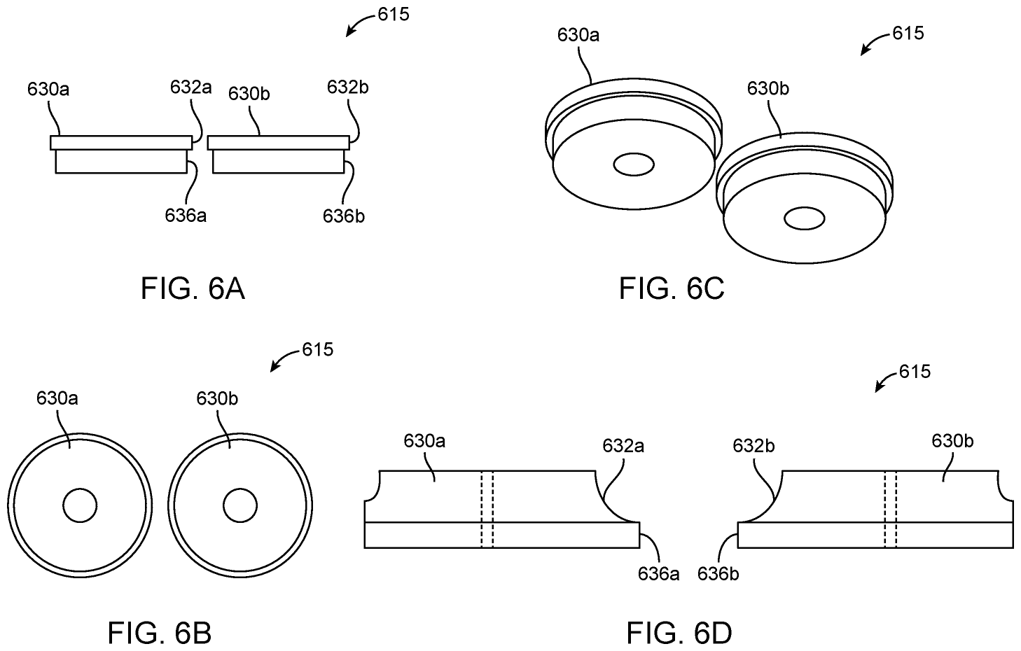

The cassette 605 can include two sets of rollers to engage a single catheter having different diameters along its length. For example, the first pair of rollers 615a can engage the larger dimension distal luminal portion 222 and the second pair of rollers 615b can engage the smaller dimension proximal control element 230. The soft elastomer can be configured to engage the different diameters of the catheter. Alternatively, the cassette can have a dedicated set of rollers that is configured to engage with the catheter 200 and automatically adjust to the different dimensions along a length of the catheter as it is advanced into and out of the patient. One or both of the rollers of a set of rollers can have two different diameters and the roller(s) could be moved relative to an external surface of the catheter (e.g., up and down or side to side) in order to match the change in diameter with the corresponding region of the catheter. FIGS. 6A-6D are various views of an implementation of a set of rollers 615 having a first tire 630a and a second tire 630b adjacent to one another so that their inner-facing surfaces are spaced a distance apart. The inner-facing surfaces can have a geometry that creates various sized gaps between the tires 630a, 630b. An upper region 632a of the first tire 630a and the corresponding upper region 632b of the second tire 630b can define a first space between them that corresponds to a shape of at least a portion of the catheter, such as a hemi-cylindrical-shaped space. This hemi-cylindrical shaped space can have an inner diameter that accommodates an outer diameter of the distal luminal portion 222 of the catheter. A lower region 636a of the first tire 630a and the corresponding lower region 636b of the second tire 630b can define a second space that corresponds to a shape of at least another portion of the catheter. The second space can be smaller in diameter compared to the first space so as to engage with the smaller dimensioned proximal control element 230 of the catheter 200.

The set of rollers 615 also can be spring-loaded to accommodate different outer diameters. The catheter 200 can include a larger dimension distal luminal portion 222 and a smaller dimension proximal control element 230. The set of rollers 615 can be configured to move away from one another so as to engage with and drive the distal luminal portion 222 during a first phase of engagement. The rollers 615 can then spring back toward each other once the distal luminal portion 222 is fully inserted through the hub 434 so that the rollers 615 can engage with and drive the proximal control element 230 during a second phase of engagement. As discussed elsewhere herein, the proximal control element 230 of the catheter 200 can be a hypotube, ribbon, or wire and have a significantly smaller outer diameter compared to the distal luminal portion 222 of the catheter 200. As an example, the distal luminal portion 222 of the catheter 200 can have an outer diameter that is sized to insert through a sheath that is 6-8 Fr, for example, 0.080″ up to about 0.105″. The proximal control element 230 of that same catheter can have an outer diameter that is much smaller, for example, about 0.014″-0.022″. Similarly, the inner navigation catheter 300 can undergo a change in outer diameter along its length from a larger outer diameter at a more distal location (e.g., about 0.080″) to a smaller outer diameter at a more proximal location (e.g., about 0.062″ to as small as about 0.022″). The catheters driven by the robotic drive system can also be different sizes and thus have different outer diameters. The robotic drive system can be used with smaller catheters (e.g., 3Fr) as well as larger catheters (e.g., 8Fr). It is useful for the feed driving mechanism to accommodate these different catheter sizes while still providing an adequate grip. The set of rollers 615 can automatically adjust their spacing so that they can accommodate the greater outer diameter of distal region of the catheter 200 and smaller outer diameter of proximal region of the catheter 200. Similarly, if the inner navigation catheter 300 changes in outer diameter along its length (e.g., incorporates a proximal step-down in outer diameter), the set of rollers 615 engaged with and configured to drive the navigation catheter 300 can automatically adjust its spacing to accommodate the change in dimension as the catheter 300 is advanced further into a patient.

Aspiration System

As mentioned with respect to FIG. 2 above, the robotic drive system 600 can be in communication with one or more other medical systems 800, such as an aspiration system 805, or can have an aspiration system integrated within the robot. Aspiration can be automated automatically turned on, off, up, down, and/or cycled faster or slower depending on the stage of a procedure or an event that triggers the change in aspiration. One or more sensors 625 can be included in the system 600 that senses pressure and/or flow at the arm 412 of the RHV 434 of the sheath. The sensors 625 can be in line with the aspiration tubing connected to a pump or canister of the aspiration system 805. A proximal pressure transducer can be position at this location and a distal pressure transducer positioned near a distal opening of a catheter 200 (e.g., about 1-5 cm from the opening). The distal pressure transducer can be arranged to read arterial pressure and the proximal pressure transducer can be arranged to read vacuum pressure. A clot corked on a distal end of the catheter 200 or progressing slowly through the lumen of the catheter would be detected. The inline pressure transducer can also be designed to detect when the fluid chamber for the exhaust side of the aspiration cycle is filled. Low positive or zero pressure at the proximal transducer can trigger software of the control system 700 to display a reminder to an operator that the fluid chamber needs filling.

In some implementations, the aspiration system 805 can include a vacuum source that couples to the catheter system via a vacuum line. The vacuum source can vary in its configuration. The vacuum source can be an active source of aspiration, such as an aspiration pump, a regular or locking syringe, a hand-held aspirator, hospital suction, or the like, configured to draw suction through the working lumen of the base sheath. As described above, the RHV of the base sheath may be sealed and aspiration initiated via a side arm of the RHV. The side arm of the RHV can be coupled to any of a variety of vacuum sources. In a preferred implementation, the vacuum source is an aspiration pump, such as the Gomco 405 Tabletop Aspirator (Allied Healthcare Products, Inc., St. Louis, MO). The aspiration pump may incorporate a programmable pump motor, such as motor controlled by a pulse width modulation, a brushless motor and controller or similar controllable motors. Alternatively, a controllable valve may be used to conduct the cyclic aspiration as described herein. In another implementation the vacuum source is a locking syringe, such as a VacLok type syringe.

Material aspirated from the catheter system can be collected within a proximal vacuum canister connected to the vacuum line. In some implementations, the proximal vacuum canister can itself be the vacuum source, such as a barrel of a locking syringe. In other implementations, the proximal vacuum canister is coupled to the vacuum source via tubing.

During a procedure when the distal tip of the catheter 200 is near or at the face of the occlusion, an operator may open the connection to the vacuum source of the aspiration system 805 (e.g., aspiration syringe). This allows for a maximum communication of aspiration force being applied through the working lumen of the sheath 400 and any catheter extending through the sheath 400 that, in turn, is in communication with the vessel at its distal end. In another implementation, the arm 412 can be connected to the vacuum source of the aspiration system 805, such as a pump. The vacuum source can provide a cyclic level of aspiration force, for example, an aspiration force that cycles between a high level of vacuum to a lower level of vacuum at a set frequency or frequencies, or from a high level of vacuum to no vacuum, or from a high level of vacuum to a positive pressure. A cyclic aspiration mode may provide a jackhammer type force on the occlusion and increase the ability to aspirate the occlusion through the catheter. One effect of these forces is to fatigue and fracture the occlusion that is often engulfed in the catheter and recruit a slow progressive movement of the thrombotic material down the length of the catheter, especially when static aspiration is incapable of doing so. Another effect of cyclic forces is to change the friction regime and to challenge connections between the clot and the vessel wall, encouraging the clot to loosen to propel the clot towards the vacuum source.

The cyclic aspiration force may be enabled through the use of one or more valves positioned between the vacuum source and the catheter system. The valve can control patency between the vacuum source, the catheter tip, and an external environment that provides the pressure differential. The valve can establish open continuity between the vacuum source, the catheter tip, and the external environment (e.g., air) when the valve is opened. Temporarily closing the valve to the external environment (e.g., manually, mechanically, or via software programming on a pump or within the controller of the robotic system) can allow the vacuum source to establish a vacuum at the catheter tip whereas subsequently opening the valve to the external environment diminishes the vacuum at the catheter tip in a controlled manner. Repeatedly opening and closing the valve develops cyclic pressure profiles at the catheter tip. Changes in the operation of the valve produces different cyclic aspiration solenoid valves, spring-operated pressure relief valve, electronically-controlled valves, manual or mechanical valves, a programmable pump motor controller, or the like. In an implementation, cyclic aspiration is applied only when clogged or restricted flow is detected in the aspiration line, either through low flow or through high vacuum, and at other times, the vacuum source reverts to a low level of flow, or be turned off. This configuration may be controlled by the user, or controlled automatically via a feedback loop to the vacuum source.

The cyclic aspiration can involve fast pressure cycling, for example, between 1 Hz and 100 Hz. The cycling can be performed at a single frequency, multiple frequencies, a dynamic array or recipe of frequencies, and for extended periods of time. A pressure cycling mechanism, such as a solenoid valve, in or near the vacuum line to the catheter, to work in tandem with an aspiration pump to apply fast oscillations in pressure. These pressure oscillations, or cycles, improve the clot removal capability of aspiration catheters and, in general, the pressure oscillating device is most effective if the distance between the pressure oscillating device and the catheter tip is minimized as pressure losses are minimized. Because the cyclic aspiration system hinges around an independent pressure oscillating mechanism it can be easily integrated into current aspiration thrombectomy systems without the need to replace the entire or existing vacuum pump assembly.

In an implementation, the cyclic frequency can be between 1 Hz and 50 Hz, and more preferably between 2 Hz and 10 Hz. In an implementation, the cyclic pressure profile includes a fast “burst” of higher frequency cycles within the frequency range (e.g. approximately 10 Hz) followed by a section of either static aspiration or slow “recovery” frequency cycles within the frequency range (e.g. approximately 3 Hz). The static or slow cyclic profile following the period of higher frequency cycles allows for building up pressure to a maximum operating pressure after pressure deteriorates at the high-speed cycling. For manual systems, aspiration cycles can include cycling between −23.8 inHg and 0 inHg at 1 Hz; cycling between −22 inHg and 0 inHg at 2 Hz; and cycling between −18.9 inHg and −5.0 inHg at 6.3 Hz. Cycling can be in the range of between 1 Hz and 3 Hz or between 1 Hz and 6 Hz, or between 1 Hz and 10 Hz, or between 1 Hz and 50 Hz. Generally, cycling can be below frequencies that increase the risk of compromising a vessel wall or increasing hemorrhage or thermal tissue damage. Low frequency mechanical perturbations should not exceed local elastic limits.

The cycling profiles can vary. The cycle aspiration time can be about 100-260 ms. Total aspiration time for both aspiration and relief for the cycle can be about 6-8 seconds. The time per cycle of relief between aspiration can be about 40-70 ms. The static aspiration between the aspiration cycles can be about 2-4 seconds.

Once corking occurs and the operator deems it necessary to withdraw the corked catheter, the operator can switch to static full pressure aspiration. Generally, an occlusion in a corked or restricted flow state where the portion of the occlusion external to the lumen may fragment and embolize due to catheter movement is withdrawn with the catheter under static aspiration and preferably under maximum force to compress and hold the embolus during catheter removal. As another example, a surgeon can apply cyclic aspiration to engulf the occlusion. Cyclic aspiration can continue for as long as the surgeon desires to aspirate the engulfed embolus through the lumen. In an implementation, it may be preferred that cyclic aspiration be applied during periods of reduced catheter movement. The engulfed occlusion may be fully evacuated (i.e. clear to the proximal canister or syringe) under cyclic aspiration. The engulfed occlusion may be fully evacuated under static aspiration. The engulfed occlusion may be at least partially evacuated using cyclic and at least partially evacuated using static aspiration before ultimately being withdrawn with the catheter. The aspiration forces can be shifted in real-time during a procedure between static aspiration and cyclic aspiration. Different cycling frequencies and amplitudes can be performed depending on the diameter of the catheter being used, the location of the aspiration valve/pressure relief/pressure altering mechanism, and the degree that which pressure is relieved or changed to create a different movement of the embolus. The operator can switch from cyclic aspiration to static aspiration at any time if it is observed there is no flow and the catheter is to be withdrawn to remove the corked embolus.

The operator can initiate aspiration of the aspiration system 805 by tapping a button or icon on a user interface 720 of the control station 700 (or an operator at the operating table 7 can initiate aspiration directly on a user interface of the robotic drive system 600), which will be described in more detail below. Alternatively, there can be a control system for aspiration system 805, a separate controller 610 for the robotic drive system 600. One or both control systems also can be connected to and operated remotely by remote control system 700. The operator may have the option to select the type of aspiration applied (e.g., static, cyclic). Selecting cyclic aspiration may also prompt a user to select (e.g., increase or decrease) the cycling frequency. Once aspiration is initiated by a user, the software running on the controller 710 can perform checks for system pressure and flow. If flow through one or more of the catheters 200 is not detected and the vacuum pressure is above a threshold pressure, the aspiration pump can be turned on (manually by an operator making a selection at the GUI 720 or automatically by software running on the controller 710). The software running on the controller 710 can continue to monitor the system pressure until a desired vacuum pressure is reached. Once the desired vacuum pressure is reached and there is no flow between the catheter 200 and the vacuum chamber of the aspiration system 805, the system can open a valve between the catheter 200 and the vacuum chamber. Flow detected when the valve between the vacuum chamber and the catheter 200 is closed indicates a leak in the system and an error message can be displayed on the GUI 720. While the valve is open between the catheter 200 and the vacuum chamber, pressure and flow can be monitored by the system to assess whether the parameters are within a specified operating range. Excessively high flow and/or pressure outside the specified operating range indicates no clot is being aspirated, but rather only blood is being aspirated from the patient, which is a safety risk, or there is a system leak. In the instance where only blood is being aspirated from the patient, software running on the controller 710 can be programmed to automatically close the valve between the catheter 200 and the vacuum chamber as well as display an error message on the GUI 720. The error message can provide information suggesting an operator advance the catheter 200 more distally inside the patient. The operator may advance the catheter 200 using one or more inputs 725 on the GUI resulting in one or more sets of rollers 715 to drive the catheter 200 distally a distance. In the instance where there is a system leak, an error message can be displayed instructing the operator to check system connections.

High vacuum pressure at the proximal end transducer in combination with low flow identified by the distal end transducer can indicate a corked clot or excessively slow passage of a clot through the catheter 200. This can also indicate that aspiration was initiated by an operator prior to withdrawal of the inner navigation catheter 300. The software running on the controller 710 an be programmed to display an error message after a certain time period of the high vacuum pressure/low flow conditions (e.g., after 30 seconds to about 2-3 minutes). This allows a sufficient time period for the clot to pass through the catheter 200 prior to triggering the error message. The error message can provide additional instructions to an operator, such as a reminder to check for whether the inner navigation catheter 300 is present, recommendation for switching to cyclic aspiration (if presently in static), or removal of the catheter 200 in order to flush out a corked clot.

Low vacuum pressure at the proximal end transducer can indicate a system leak or no clot aspiration. Software running on the controller 710 can be programmed to close the valve to the catheter 200 and display an error message instructing an operator to check that the aspiration system 805 is properly connected and that there are no leaks.

Arterial pressure at the distal end transducer and high vacuum pressure at the proximal end transducer with no flow can indicate a corked catheter 200 where the clot has passed the distal pressure transducer and is traveling through the catheter 200 lumen with at least some success. The software running on the controller 710 can be programmed to maintain conditions for a period of time for clot aspiration to succeed before alerting an operator with an error message on the GUI 720 to recommend switching from static to cyclic aspiration or remove the catheter 200 from the patient in order to flush out the corked clot. Removal of the catheter 200 can be performed by the robotic drive system 600 upon an operator activating a particular input 725 on the GUI 720 to retract the catheter 200.

Moderate flow and pressures that remain within the selected operating range (e.g., between arterial and maximum vacuum) indicates successful aspiration of clot(s). If no error conditions are detected, the software running on the controller 710 is programmed to automatically close the valve after specified time limit (e.g., 60 seconds). Aspiration can be restarted by the operator, such as by activating an “aspiration ON” button or tapping an input on the GUI 720.

Control System/User Interface

The control system 700 of the procedure system 10 can be used by an operator to control one or more functions of the robotic drive system 600, the aspiration system 805, or other functionality. As discussed above, the control system 700 can include a computing device having a user interface 720 with one or more inputs 725 and one or more displays 730. FIG. 5 is an example graphical user interface (GUI) 720 of the control system 700 used by an operator to control the various functions of the system 10. It should be appreciated that the user interface of the robotic drive system 600 can be used by an operator to control various functions of the system 10 as well and the GUI features shown in FIG. 5 and discussed below with regard to the control system 700 can be mirrored on the user interface or controller of the system 600 located by the patient. Where control of the system 10 is described as being performed by an operator on the control system 700, an operator can also control the system 10 using the user interface of the robotic drive system 600 and need not be remote.

The relative arrangement and configuration of the inputs 725 on the GUI 720 can vary. Some of the inputs 725 can be physical components configured to be engaged by an operator (e.g., button, slider, dial, joystick, foot pedal) and other inputs 725 can be icons displayed on a screen that are tapped or selected as is known in the art.

FIG. 5 shows the information displayed can be organized into columns for each component. The information can be organized into a guidewire column 5500, navigation catheter column 5300, aspiration catheter column 5200, and a guide sheath column 5400. Each component column can have certain information and/or inputs displayed relative to the function of that component. Each component column can provide an indication of what component the information relates to, such as by labeling with text, shapes, and/or color. For example, the navigation catheter column 500 can be labeled with the words “navigation catheter” or another name that suitably identified that component and can have icons in a particular color scheme that is unique to that component. The aspiration catheter column 5200 can also be labeled with words identifying that component and by displaying icons in a different color unique to that component. FIG. 5 shows the columns arranged on the display so that the guidewire column 5500 is on the far left and the guide sheath column 5400 is on the far right. The columns can be arranged on the display in any of a variety of orders and the arrangement can be programmed by an operator at the time of a procedure. In an implementation, the columns can be arranged on the display in the same order that they are positioned in the patient and the rollers so that the manipulation of the component mirrors on the display the arrangement of manipulation at the components themselves. With regard to FIG. 4A, the guidewire rollers 615d are located at the far right or proximal side of the cassette 605 and the catheter rollers 615a, 615b are located to the left or distal side of the navigation catheter rollers 615c, which are located to the left of the guidewire rollers 615d. The sheath manipulator 620 is on the far left or distal side. The guidewire column 5500 can be arranged on the display at the far right next to the navigation catheter column 5300 and the sheath column 5400 can be arranged on the display at the far left next to the aspiration catheter column 5200. The manipulations performed by an operator on the remote control system more intuitively mirror the manipulations that are occurring within the cassette 605 for that component.

Each component column can display a movement output (5505, 5305, 5405) showing a magnitude of linear translation of the component relative to a baseline and an angle output (5510, 5310, 5410) showing angle of rotation relative to a baseline, if applicable, and a corresponding reset input. Some components, such as the aspiration catheter, may have additional information display, such as a vacuum pressure output 5215, showing vacuum pressure within the catheter or a flow rate output 5220 showing flow rate within the catheter. The guide sheath column 5400 may include a movement output 5425 displaying the movement of the guide sheath from the original placement in the patient.

Still with respect to FIG. 5, each component column may also include one or more inputs (5530, 5330, 5230, 5430) to incrementally increase or decrease movement and/or angle, and others. Additional inputs include a rotate input 5535, an oscillation input 5540, and a cycle aspiration input 5545. For example, toggling the oscillation input 5540 to “on” can intermittently retract-advance-retract the catheter 200 to mitigate the occurrence of corking. Another input can include a link input 5565 that joins the set of rollers 615 for the navigation catheter 300 to the aspiration catheter 200 so that the two components are moved synchronously. The input can also indicate that there are two components that are mechanically linked, such as with a physical clip, to move synchronously. An operator upon the components being clipped together can toggle the input to ensure the operator is alerted to how the components will move together upon motion of a particular set of rollers. Each component can include an input that is a toggle switch 5550 configured to move devices distally by toggling the switch forward or move proximally by toggling the switch backward. In some cases the switch 5550 can be a joystick capable of being twisted to rotate one or more of the components. The switch 5550 for the aspiration catheter 200 can additionally include a button 5555 on top of the switch 5550 that allows for an operator to manually actuate “aspiration ON” and/or “aspiration OFF” with a button press. Alternatively, “aspiration OFF” can be automatically initiated, such as by software running on the controller 710 as described elsewhere herein.

The location and configuration of the switches 5550 for the various components can provide information to the user regarding which component of the system is being manipulated upon actuation of that switch 5550. For example, the first switch 5550 can be a first color and the second switch 5550 can be a second, different color. The colors can coordinate with color on the GUI showing one or more indicators for that component. The position of the joysticks on the console can also coordinate with the position of the indicators on the GUI. The degree of change provided by the switches 5550 upon actuation can be selected or programmed by a user depending on the incremental change in position desired with each actuation.

The one or more outputs can include alerts to provide the operator with information regarding status of the robotic drive system 600 or other component of the system 10. The alerts can be auditory, visual, and/or tactile signals. A visual signal can include one or more LEDs, icons on the display, or other signal. The auditory signal can be one or more beeps that indicate information to a user. The alerts can be informative, such as speed to advancement represented by a frequency of a sound. The one or more outputs can also include alarms (auditory, visual, and/or tactile signals) that can indicate a malfunction, interruption of communication between the control system 700 and the robotic drive system 600, patient condition change, etc. An alert may be cleared by a user whereas an alarm will continue to be triggered until the problem is resolved. The visual signal can include a display of the status of components of the robotic system. For example, a light of a first color (e.g., red) can be displayed if a component of the distal access system 100 is not loaded and/or latched in the associated cassette position and a second color (e.g., green) can be displayed once the component is correctly loaded and/or latched. The visual signal need not be a color and can incorporate a symbol and/or text providing information about loading and/or latching.

The oscillation input 5540 parameters can be programmed by a user to cause the support catheter to follow a pattern of one or more retractions and one or more advancements. In an implementation, actuating the oscillation input 5540 can initiate a pattern as follows 1) a period of static aspiration while the catheter distal opening is at a first position relative to an occlusion; 2) a short retraction of the catheter to place the distal opening at a second position relative to the occlusion, and 3) advancement of the catheter to place the distal opening back to the first position. The period of static aspiration can be about 5 seconds of static aspiration although this can vary anywhere from 1 second up to about 30 seconds. The short retraction of the catheter following the static aspiration can also vary including about 1 mm-50 mm, about 2 mm-25 mm, about 3 mm-10 mm, or preferably about 5 mm. The advancement of the catheter to place the distal opening back to the first position can also advance the catheter to a location that is further distal to the first position (e.g., if at least some of the occlusion has been engulfed) or further proximal to the first position. The preferred advancement is to a position in which the distal end of the catheter is in contact with the occlusion.

Methods of Use

Below are examples of methods of using of the systems described herein for performing a neurovascular procedure using a distal vascular access system 100 and a robotic drive system 600. Included are examples of performing aspiration through the catheter 200 and guide sheath 400 using an aspiration system 805. Other neurovascular procedures are considered as well including delivery of PTA catheters and implantation of expandable tools including coils, stents, or flow diverters.

In an implementation of performing a robot-assisted interventional procedure on a patient in the neurovasculature, an introducer sheath and/or guide sheath, such as guide sheath 400, is used to gain access to the vasculature of a patient, for example, at a femoral artery or radial artery access site. A guidewire and catheter system can be introduced through the sheath into the patient vasculature. The catheter system 150 can include a support catheter 200 and a navigation catheter 300. The catheter system 150 can be advanced through the sheath 400 as described elsewhere herein. If desired, the guidewire and catheters 200, 300 of the catheter system 150 can be advanced to a first stage manually by a user, for example, from the access site to the distal end region of the guide sheath 400. The catheter system can then be coupled to the robotic drive system 600, such as at a cassette 605. The robotic drive system 600 is operatively coupled to a controller 610 and/or remote controller 710 operable by inputs from an operator. The robotic drive system 600 includes a plurality of rollers 615 or other actuator bodies that are movable in response to operator inputs. The support catheter 200 includes a distal luminal portion 222 having a distal opening 231 and a proximal opening 242. A single lumen 223 extends between the proximal opening 242 and the distal opening 231. The support catheter 200 also includes a proximal control element 230 coupled to the distal luminal portion 222 near the proximal opening 242. The proximal control element 230 is without a lumen extending through it. The navigation catheter 300 includes a guide wire lumen 368 and an outer diameter sized to fill the lumen 223 of the support catheter 200. The navigation catheter includes a distal tip region 346 and a proximal extension. The distal tip region can taper from the outer diameter to a distal-most end defining an opening from the guide wire lumen 368. The navigation catheter 300 is axially positionable through the single lumen 223 of the support catheter 200 so that the distal tip region 346 of the navigation catheter 300 is extendable from the distal opening 231 of the distal luminal portion 222 of the support catheter 200. The support catheter 200 having the navigation catheter 300 positioned within its single lumen 223 is navigable to a vessel that is distal to a petrous portion of an internal carotid artery (ICA).

The method further includes coupling the proximal control element 230 of the support catheter 200 to a first set of rollers 615 of the plurality of rollers 615 and coupling the proximal extension 366 of the navigation catheter 300 to a second set of rollers 615 of the plurality of rollers 615. The second set of rollers 615 are located proximal of the first set of rollers 615. The method further includes moving the first and second sets of rollers 615 in at least one degree of freedom in response to operator inputs.

The method optionally includes manipulating the guide sheath 400 with control system 600, for example, with a sheath hub housing moveably controlled in at least one degree of freedom in response to operator inputs. FIG. 4C illustrates an implementation of providing independent movement of the guide sheath 400 relative to other components coupled to the cassette 605. The proximal end of the guide sheath 400, such as the RHV 434, can be positioned within a rack 640 having an external surface arranged to engage with teeth on a small gear or pinion 642. The teeth on the pinion 642 mesh with corresponding features on the external surface of the rack 640. The rack 640 can be floating and the pinion 642 carried on bearings so that the rack 640 translates axially along the longitudinal axis A as the pinion 642 rotates. The guide sheath 400 can be advanced manually to a distal location. For example, the tip of the guide sheath 400 can be advanced to the common carotid artery, the cervical ICA, or a more distal section of the ICA for carotid artery or anterior circulation procedures, or subclavian artery or vertebral artery for posterior circulation procedures. This initial stage of advancement can be performed manually by an operator until the desired location is reached prior to coupling the hub to the rack 640. The sheath can move along with the rack 640 for limited axial adjustments of the sheath independently of the other components coupled to the cassette 605.

The distal luminal portion 222 of the catheter 200 having an outer diameter sized to be positioned within a working lumen of a guide sheath 400 is advanced through the guide sheath 400 coupled to the robotic drive system 600 configured to drive the catheter 200.

In a method involving aspiration of clot, the guide sheath 400 is operatively coupled to an aspiration system 805. The catheter 200 and navigation catheter 300 can be advanced to a location of a clot blockage. The navigation catheter 300 can be removed and aspiration of the clot performed via a contiguous lumen formed from the working lumen of the guide sheath 400 and the single lumen of the catheter 200. A control station 700 controls the drive system 600 and the aspiration system 805.

In an interrelated implementation of a method of using the robotic drive system to perform a procedure, a control signal is generated that corresponds to movement of a master input device. A plurality of drive elements of an instrument driver is moved in response to the control signal to translate a catheter system operatively coupled to the plurality of drive elements. The catheter system has a distal end inserted in a vessel in a patient. The catheter system includes a support catheter having a distal luminal portion coupled at a proximal end region to a proximal control element adjacent a proximal opening from a single lumen of the distal luminal portion. The proximal control element is engaged by a first drive element. The catheter system further includes a navigation catheter having a guide wire lumen and a distal tip region that tapers from an outer diameter sized to fill the single lumen of the support catheter to a distal-most end defining an opening from the guide wire lumen. A proximal extension of the navigation catheter is engaged by a second drive element. Movement of the plurality of drive elements causes a corresponding movement of the catheter system. The control signal causes individual ones of the plurality of drive elements to move independently of one another in order to achieve a desired advancement of the catheter system in the vessel and relative extension of the navigation catheter to the support catheter. A further control signal is generated using the master input device. A further drive element of the instrument driver moves in response to the further control signal.

In further implementations, the robotic drive system 600 can be used to perform high-speed operation techniques. The catheter system including a support catheter 200 and a navigation catheter 300 can be inserted so that a distal end of the navigation catheter 300 (i.e. extending distal to the distal end of the support catheter 200) can be positioned from outside the body to a location near the distal end of the sheath 400 using the robotic drive system 600. The robotic drive system 600 can advance the catheter system to this location relative to the sheath 400 automatically at a robot-controlled high speed and then automatically stopped once reaching the target location near the distal end of the sheath 400. The operator can then control speed of advancement of the catheter system 150 outside the sheath 400 using the control system 700. The robot-controlled high speed for advancement of the catheter system 150 through the sheath 400 can be about at least about 1 cm per second, preferably at least about 5 cm per second up to about 10-15 cm per second, although other speeds are considered.

The robotic drive system 600 can automatically control withdrawal of the navigation catheter 300 at a robot-controlled high speed to create the piston effect as described elsewhere herein. The robot-controlled high speed for withdrawal of the navigation catheter 300 from the support catheter 200 can be between about 4 cm per second up to about 165 cm per second, or about 10 cm per second up to about 75 cm per second, or about 20 cm per second to about 25 cm per second.

The robotic drive system 600 can automatically control withdrawal of the support catheter 200 following a procedure. The robot-controlled high speed for withdrawal of the support catheter 200 away from the procedure site can be at least about 1 cm per second, preferably at least about 5 cm per second up to about 10-15 cm per second, although other speeds are considered. If the catheter 200 is corked the withdrawal of the catheter 200 may be slower (e.g., 10-15 cm/second) whereas if the catheter 200 has fully ingested the clot and free flow of blood is observed through aspiration tubing, the pump might be turned off and the catheter removed more quickly (e.g., 15-20 cm/second).

The robotic drive system can be capable of any of a variety of speeds well outside these specified ranges. The user can selected minimum and/or maximum thresholds the system is capable of upon actuation whether the actuation is for advancement of the system or withdrawal of one or more of the components. Additionally, the robotic drive system can incorporate a feedback loop between the drives system and one or more sensors of the system (e.g., a sensor monitoring blood flow through the tubing). The feedback can change a speed of advancement or withdrawal including completely stopping all movements. The robot-controlled high speed advancement and withdrawal can have an emergency shut-off, for example, if a sensor senses a problem. For example, a force sensor can be incorporated that can sense resistance of one or more of the catheter system components outside of an acceptable range and automatically shut-off motion of that component driven by the rollers 615.

Materials

One or more components of the catheters described herein may include or be made from a variety of materials including one or more of a metal, metal alloy, polymer, a metal-polymer composite, ceramics, hydrophilic polymers, polyacrylamide, polyethers, polyamides, polyethylenes, polyurethanes, copolymers thereof, polyvinyl chloride (PVC), PEO, PEO-impregnated polyurethanes, such as Hydrothane, Tecophilic polyurethane, Tecothane, PEO soft segmented polyurethane blended with Tecoflex, thermoplastic starch, PVP, and combinations thereof, and the like, or other suitable materials.

Some examples of suitable metals and metal alloys include stainless steel, such as 304V, 304L, and 316LV stainless steel; mild steel; nickel-titanium alloy, such as linear-elastic and/or super-elastic nitinol; other nickel alloys, such as nickel-chromium-molybdenum alloys (e.g., UNS: N06625 such as INCONEL® 625, UNS: N06022 such as HASTELLOY® C-22®, UNS: N10276, such as HASTELLOY® C276®, other HASTELLOY® alloys, and the like), nickel-copper alloys (e.g., UNS: N04400, such as MONEL® 400, NICKELVAC® 400, NICORROS® 400, and the like), nickel-cobalt-chromium-molybdenum alloys (e.g., UNS: R30035, such as MP35-N® and the like), nickel-molybdenum alloys (e.g., UNS: N10665, such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003, such as ELGILOY®, PHYNOX®, and the like); platinum enriched stainless steel; titanium; combinations thereof; and the like; or any other suitable material and as described elsewhere herein.

Inner liner materials of the catheters described herein can include low friction polymers, such as PTFE (polytetrafluoroethylene) or FEP (fluorinated ethylene propylene), PTFE with polyurethane layer (Tecoflex). Reinforcement layer materials of the catheters described herein can be incorporated to provide mechanical integrity for applying torque and/or to prevent flattening or kinking, such as metals including stainless steel, Nitinol, Nitinol braid, helical ribbon, helical wire, cut stainless steel, or the like, or stiff polymers, such as PEEK. Reinforcement fiber materials of the catheters described herein can include various high tenacity polymers like Kevlar, polyester, meta-para-aramide, PEEK, single fiber, multi-fiber bundles, high tensile strength polymers, metals, or alloys, and the like. Outer jacket materials of the catheters described herein can provide mechanical integrity and can be contracted of a variety of materials, such as polyethylene, polyurethane, PEBAX, nylon, Tecothane, and the like. Other coating materials of the catheters described herein include paralene, Teflon, silicone, polyimide-polytetrafluoroetheylene, and the like. The inner liner may further include different surface finishes, such as dimples, bumps, ridges, troughs. The surface finishes may be randomly disposed, linearly disposed, spirally disposed, or otherwise disposed using a specific pattern along the length of the catheter. It is further contemplated that the inner liner may include a mixture of different surface finishes, for example, one section may have dimples, another section may have troughs, etc. Additionally, the surface finish may be incorporated along the entire length of the catheter or only in sections of the catheter. It is also contemplated that the inner liner may further include an electrosprayed layer, whereby materials could be incorporated into the inner liner. Examples of materials can include low friction materials as described above. Alternatively, the electrosprayed or electrospun layer may incorporate a beneficial agent that becomes free from the coating when exposed to blood, or to compression from a clot, for example, the beneficial agent may be a tissue plasminogen activator (tPA) or heparin encased in alginate.

The cassette may be designed for single use and be disposable and replaced. The cassette also may be formed from materials that are configured to be sterilized and reused. The cassette can be formed of plastic, such as injection molded plastics or 3D printed plastic one or more portions being transparent or translucent. The material of one or more regions of the cassette may vary including plastics, such as polycarbonate, acrylonitrile butadiene styrene (ABS), acrylic, nylon polyamide, polyethylene, polypropylene, polystyrene, and also metals, such as stainless steel. One or more portions of cassette may also be machined.

The cassette may be packaged individually and sterilized, such as by Ethylene oxide or radiation. The catheter systems disclosed herein may be packaged together in a single package, where the support/aspiration catheters and corresponding navigation catheters are packaged in a coil tube. The finished package would be sterilized using sterilization methods, such as Ethylene oxide or radiation and labeled and boxed. Instructions for use may also be provided in-box or through an internet link printed on the label.

Implementations describe catheters and delivery systems and methods to deliver catheters to target anatomies. However, while some implementations are described with specific regard to delivering catheters to a target vessel of a neurovascular anatomy, such as a cerebral vessel, the implementations are not so limited and certain implementations may also be applicable to other uses. For example, the catheters can be adapted for delivery to different neuroanatomies, such as subclavian, vertebral, carotid vessels as well as to the coronary anatomy or peripheral vascular anatomy, to name only a few possible applications. Although the systems described herein are described as being useful for treating a particular condition or pathology, that the condition or pathology being treated may vary and are not intended to be limiting.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator or site of insertion such that the terms “proximal” and “distal” are in reference to an operator or site of insertion using the device. A region of the device that is closer to an operator or site of insertion may be described herein as “proximal” and a region of the device that is further away from an operator or site of insertion may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the catheters and/or delivery systems to a specific configuration described in the various implementations.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

What is claimed is:

1. A robotic procedure system for treating neurovasculature of a patient, the system comprising:
a guide sheath comprising:
a sheath body having at least one lumen extending between a proximal end region and a distal end region defining a distal opening from the at least one lumen; and
a hub coupled to the proximal end region of the sheath body;
a catheter system comprising:
a support catheter comprising a distal luminal portion having an outer diameter sized to be positioned within the at least one lumen of the guide sheath, the distal luminal portion coupled at a proximal end region to a proximal control element adjacent a proximal opening from a single lumen of the distal luminal portion; and
a navigation catheter having a guidewire lumen, a distal tip region that tapers from an outer diameter sized to fill the single lumen of the support catheter to a distal-most end defining an opening from the guidewire lumen, and a proximal extension; and
a robotic drive system configured to drive the catheter system within a patient's vessel, the robotic drive system comprising:
a cassette having at least a first set of rollers and at least a second set of rollers, the first set of rollers configured to engage the proximal control element of the support catheter and the second set of rollers configured to engage the proximal extension of the navigation catheter, the second set of rollers positioned proximal of the first set of rollers; and
a controller operatively coupled to the cassette, the controller configured to control the first set and second set of rollers so as to determine a magnitude of linear translation of the support catheter and a magnitude of linear translation of the navigation catheter.

2. The system of claim 1, further comprising a guidewire and a third set of rollers located proximal to the second set of rollers that is configured to engage the guidewire, the spacing of the third set of rollers designed to allow a full range of motion of the navigation catheter through the second set of rollers.

3. The system of claim 1, wherein the proximal control element of the support catheter is a ribbon, a hypotube, or a solid round wire.

4. The system of claim 1, wherein the proximal extension of the navigation catheter is a polymer-coated rigid component.

5. The system of claim 1, wherein at least one of the first and second set of rollers is configured to accommodate different outer diameters.

6. The system of claim 1, wherein rollers of the first set of rollers are spaced closer together than rollers of the second set of rollers.

7. The system of claim 1, wherein the first set of rollers is proximal to and off-set from an axis of the guide sheath working lumen.

8. The system of claim 1, further comprising an aspiration system operatively coupled to the controller.

9. The system of claim 8, wherein the aspiration system is configured to apply static or cyclic aspiration.

10. The system of claim 9, wherein the cyclic aspiration is applied using a spring-operated pressure relief valve or a solenoid valve.

11. The system of claim 9, wherein the aspiration system is actuated manually or by software running on the controller.

12. The system of claim 1, wherein the guide sheath is coupled to the robotic drive system by securing the hub to the cassette via at least one connector and/or cavity within the cassette.

13. The system of claim 12, wherein the at least one connector is configured to rotate the guide sheath around a longitudinal axis of the sheath body.

14. The system of claim 1, wherein at least one of the first set and the second set of rollers is configured change the magnitude of linear translation, an angle, or both.

15. The system of claim 1, wherein the first set of rollers and the second set of rollers are configured to be driven in unison.

16. The system of claim 15, wherein the first set of rollers and the second set of rollers are driven in unison due to a mechanical linkage.

17. The system of claim 1, further comprising one or more markers on the support catheter.

18. The system of claim 17, further comprising one or more markers on the navigation catheter.

19. The system of claim 18, wherein the controller is programmed to detect the one or more markers on the support catheter and the one or more markers on the navigation catheter to assess extension of the support catheter relative to the navigation catheter.

20. The system of claim 18, wherein the controller is programmed to detect the one or more markers on the support catheter and the one or more markers on the navigation catheter to assess total distance of advancement.

21. The system of claim 8, further comprising one or more flow sensors and one or more pressure transducers.

22. The system of claim 1, further comprising an oscillation input configured to cause the support catheter to follow a pattern of one or more retractions and one or more advancements.

23. The system of claim 22, wherein the oscillation input is programmable by a user.

24. The system of claim 22, wherein the oscillation input initiates a pattern of a short retraction of the support catheter withdrawing a distal opening of the distal luminal portion from a first position relative to an occlusion to a second position relative to the occlusion and an advancement of the support catheter advancing the distal opening from the second position towards the first position.

25. The system of claim 24, wherein the pattern begins after a period of static aspiration through the support catheter.

26. A method for performing robotic surgery on a patient in the neurovasculature, the method comprising:
coupling a catheter system to a robotic drive system, the robotic drive system operatively coupled to a controller operable by inputs from an operator, the robotic drive system having a plurality of rollers movable in response to operator inputs, the catheter system comprising:
a support catheter comprising:
a distal luminal portion having a distal opening and a proximal opening, a single lumen extending between the proximal opening and the distal opening; and
a proximal control element without a lumen coupled to the distal luminal portion near the proximal opening; and
a navigation catheter comprising:
a guidewire lumen;
an outer diameter sized to fill the single lumen of the support catheter;
a distal tip region; and
a proximal extension,
wherein the navigation catheter is axially positionable through the single lumen of the support catheter so that the distal tip region of the navigation catheter is extendable from the distal opening of the distal luminal portion of the support catheter, and
wherein the support catheter having the navigation catheter positioned within the single lumen is navigable to a vessel distal to a petrous portion of an internal carotid artery;
coupling the proximal control element of the support catheter to a first set of rollers of the plurality of rollers;
coupling the proximal extension of the navigation catheter to a second set of rollers of the plurality of rollers, the second set of rollers located proximal of the first set of rollers; and
moving the first and second sets of navigation rollers in at least one degree of freedom in response to operator inputs.

27. A robotically controlled procedure system for removing a clot from a patient, the system comprising:
a guide sheath having at least one working lumen;
a catheter system including a catheter comprising a distal luminal portion having an outer diameter sized to be positioned within the working lumen, the distal luminal portion coupled at a proximal end region to a proximal control element adjacent a proximal opening from a single lumen of the distal luminal portion;
wherein the guide sheath is operatively coupled to an aspiration system for performing an aspiration function on the clot via a contiguous lumen formed from the working lumen of the guide sheath and the single lumen of the catheter;
an instrument drive system for driving the catheter system, wherein the instrument drive system comprises a first instrument driver for driving the catheter and a second instrument driver for driving an interventional device; and a remote control station for controlling the instrument drive system and the aspiration system.

28. A robotic catheter system, comprising:
a controller including a master input device configured for operation by a physician at a location remote from a patient;
an instrument driver in communication with the controller, the instrument driver comprising a housing having a catheter interface movable relative to the housing, the catheter interface including a plurality of catheter drive elements coupled to respective motors located within the housing, the motors responsive to control signals generated, at least in part, by movement of the master input device for actuating the catheter drive elements and movement of the catheter interface relative to the housing; and
a catheter having a proximal ribbon coupled to a distal luminal portion having a single working lumen, the proximal ribbon operatively coupled to the catheter interface, the catheter axially moveable relative to a guide sheath such that movement of the catheter may be controlled by the master input device.
